(19) United States
(12) Patent Application Publication
(10) Pub. No.: US 2026/0248439 A1
(43) Pub. Date: Aug. 27, 2026

(54) QRS DETECTION AND BRACKETING

(71) Applicant: MEDTRONIC, INC., Minneapolis, MN (US)

(72) Inventors: Trent Fischer, St. Paul, MN (US); Karen Kleckner, Minneapolis, MN (US); Daniel S. Flo, Minneapolis, MN (US); Marc C. Steckler, Circles Pines, MN (US); Subham Ghosh, Minneapolis, MN (US)

(21) Appl. No.: 19/649,605

(22) Filed: Apr. 16, 2026

Related U.S. Application Data
(62) Division of application No. 17/320,778, filed on May 14, 2021, now Pat. No. 12,605,103.
(60) Provisional application No. 63/028,293, filed on May 21, 2020.
(60) Provisional application No. 63/034,397, filed on Jun. 3, 2020.

(51) Int. Cl. A61B 5/366; A61B 5/00; A61B 5/282; A61N 1/365; G16H 40/67

(52) U.S. Cl. CPC A61B 5/366; G16H 40/67; A61B 5/282; A61B 5/725; A61N 1/36507

(57) ABSTRACT

Electrical activity from tissue of a patient is monitored using a plurality of external electrodes to generate a plurality of electrical signals over time. The plurality of electrical signals are filtered using a first filter having a first frequency range to generate a plurality of first filtered signals. The plurality of electrical signals are filtered using a second filter having a second frequency range different than the first frequency range to generate a plurality of second filtered signals. At least one QRS complex is detected based on the plurality of first filtered signals. A QRS peak of the at least one QRS complex is detected based on the plurality of second filtered signals and the detected at least one QRS complex.

This application is a divisional of U.S. application Ser. No. 17/320,778, filed May 14, 2021 and which claims the benefit of U.S. Provisional Application No. 63/028,293, filed May 21, 2020 and U.S. Provisional Application No. 63/034,397, filed Jun. 3, 2020, which are incorporated herein by reference in their entireties.

The disclosure herein relates to systems and methods for use in the detecting and bracketing of QRS complexes in cardiac signals monitored by a plurality of external electrodes.

Implantable medical devices (IMDs), such as implantable pacemakers, cardioverters, defibrillators, or pacemaker-cardioverter-defibrillators, provide therapeutic electrical stimulation to the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart if arrhythmia is detected based on the intrinsic depolarizations.

IMDs may also provide cardiac resynchronization therapy (CRT), which is a form of pacing. CRT involves the delivery of pacing to the left ventricle, or both the left and right ventricles. The timing and location of the delivery of pacing pulses to the ventricle(s) may be selected to improve the coordination and efficiency of ventricular contraction.

Systems for implanting medical devices may include workstations or other equipment in addition to the implantable medical device itself. In some cases, these other pieces of equipment assist the physician or other technician with placing the intracardiac leads at particular locations on the heart. In some cases, the equipment provides information to the physician about the electrical activity of the heart and the location of the intracardiac lead. The equipment may perform similar functions as the medical device, including delivering electrical stimulation to the heart and sensing the depolarizations of the heart. In some cases, the equipment may include equipment for obtaining an electrocardiogram (ECG) via electrodes on the surface, or skin, of the patient. More specifically, the patient may have a plurality of electrodes on an ECG belt or vest that surrounds the torso of the patient. After the belt or vest has been secured to the torso, a physician can perform a series of tests to evaluate a patient's cardiac response. The evaluation process can include detection of a baseline rhythm in which no electrical stimuli is delivered to cardiac tissue and another rhythm after electrical stimuli is delivered to the cardiac tissue.

The ECG electrodes placed on the body surface of the patient may be used for various therapeutic purposes (e.g., cardiac resynchronization therapy) including optimizing lead location, pacing parameters, etc. based on one or more metrics derived from the signals captured by the ECG electrodes. For example, electrical heterogeneity information may be derived from electrical activation times computed from multiple electrodes on the body surface.

Further, the signals from multiple electrodes on the body surface can be used to determine one or more specific ECG features such as, e.g., QRS onset, peak, QRS offset, etc. for a series of multiple heartbeats. Such ECG features may be used by themselves to evaluate cardiac health and/or therapy, or may be used to calculate, or compute, activation times.

### SUMMARY

The exemplary systems and methods described herein may be configured to assist users (e.g., physicians) in configuring cardiac therapy (e.g., cardiac therapy being performed on a patient during and/or after implantation of cardiac therapy apparatus). The systems and methods may be described as being noninvasive. For example, the systems and methods may not need implantable devices such as leads, probes, sensors, catheters, etc. to evaluate and configure the cardiac therapy. Instead, the systems and methods may use electrical measurements taken noninvasively using, e.g., a plurality of external electrodes attached to the skin of a patient about the patient's torso.

One exemplary system for use in cardiac evaluation may include an electrode apparatus comprising a plurality of external electrodes to be disposed proximate a patient's skin. A computing apparatus comprises processing circuitry. The computing apparatus is operably coupled to the electrode apparatus. The computing apparatus is configured to monitor electrical activity from tissue of a patient using a plurality of external electrodes to generate a plurality of electrical signals over time. The plurality of electrical signals are filtered using a first filter having a first frequency range to generate a plurality of first filtered signals. The plurality of electrical signals are filtered using a second filter having a second frequency range different than the first frequency range to generate a plurality of second filtered signals. At least one QRS complex is detected based on the plurality of first filtered signals. A QRS peak of the at least one QRS complex is detected based on the plurality of second filtered signals and the detected at least one QRS complex.

One exemplary method for use in cardiac evaluation may include monitoring electrical activity from tissue of a patient using a plurality of external electrodes to generate a plurality of electrical signals over time. The plurality of electrical signals are filtered using a first filter having a first frequency range to generate a plurality of first filtered signals. The plurality of electrical signals are filtered using a second filter having a second frequency range different than the first frequency range to generate a plurality of second filtered signals. At least one QRS complex is detected based on the plurality of first filtered signals. A QRS peak of the at least one QRS complex is detected based on the plurality of second filtered signals and the detected at least one QRS complex.

An exemplary system for use in cardiac evaluation may include an electrode apparatus comprising a plurality of external electrodes to be disposed proximate a patient's skin. A computing apparatus comprises processing circuitry. The computing apparatus is operably coupled to the electrode apparatus. The computing apparatus is configured to monitor electrical activity from tissue of a patient using a plurality of external electrodes to generate a plurality of electrical signals over time. The plurality of electrical signals are filtered using at least one filter to generate a plurality of filtered signals. A QRS peak is detected based on the plurality of filtered signals. A threshold function is determined based on the QRS peak. The threshold function is configured to provide sensitivity for detecting at least one subsequent QRS complex. The at least one QRS complex is detected based on the threshold function.

An exemplary method for use in cardiac evaluation may include monitoring electrical activity from tissue of a patient using a plurality of external electrodes to generate a plurality of electrical signals over time. The plurality of electrical signals are filtered using at least one filter to generate a plurality of filtered signals. A QRS peak is detected based on the plurality of filtered signals. A threshold function is determined based on the QRS peak. The threshold function is configured to provide sensitivity for detecting at least one subsequent QRS complex. The at least one QRS complex is detected based on the threshold function.

An exemplary system for use in cardiac evaluation may include an electrode apparatus comprising a plurality of external electrodes to be disposed proximate a patient's skin. A computing apparatus comprises processing circuitry. The computing apparatus is operably coupled to the electrode apparatus. The computing apparatus is configured to monitor electrical activity from tissue of a patient using a plurality of external electrodes to generate a plurality of electrical signals over time. The plurality of electrical signals are filtered using a first filter and a second filter to generate a plurality of first filtered signals and a plurality of second filtered signals. The first filter and the second filter have different frequency ranges. A QRS peak is detected based on the plurality of first and second filtered signals. A dispersion signal is generated from the plurality of second filtered signals. The dispersion signal is representative of the dispersion of the plurality of second filtered signals over time. A first derivative signal is determined based on the dispersion signal. A QRS onset time value and a QRS offset time value corresponding to the at least one QRS complex is determined based on the first derivative signal.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an exemplary system including electrode apparatus, display apparatus, and computing apparatus.

FIGS. 2-3 are diagrams of exemplary external electrode apparatus for measuring torso-surface potentials.

FIG. 4 shows an exemplary method for detecting QRS complexes.

FIG. 5 illustrates an exemplary method for detecting a QRS complex and at least one subsequent QRS complex based on a threshold function.

FIGS. 6A and 6B illustrate a more detailed process for detecting at least one QRS complex in accordance with embodiments described herein.

FIG. 7 shows an example threshold function in accordance with embodiments described herein.

FIG. 8 illustrates an exemplary method for determining an onset and an offset of a corresponding QRS complex.

FIGS. 9 and 10A-10C show a more detailed process for determining the onset and offset values for at least one QRS complex in accordance with embodiments described herein.

FIG. 11 is a diagram of an illustrative system including an illustrative implantable medical device (IMD).

FIG. 12A is a diagram of the illustrative IMD of FIG. 11.

FIG. 12B is a diagram of an enlarged view of a distal end of the electrical lead disposed in the left ventricle of FIG. 12A.

FIG. 13A is a block diagram of an illustrative IMD, e.g., of the systems of FIGS. 11-12.

FIG. 13B is another block diagram of an illustrative IMD (e.g., an implantable pulse generator) circuitry and associated leads employed in the systems of FIGS. 11-12).

### DETAILED DESCRIPTION OF EXEMPLARY EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

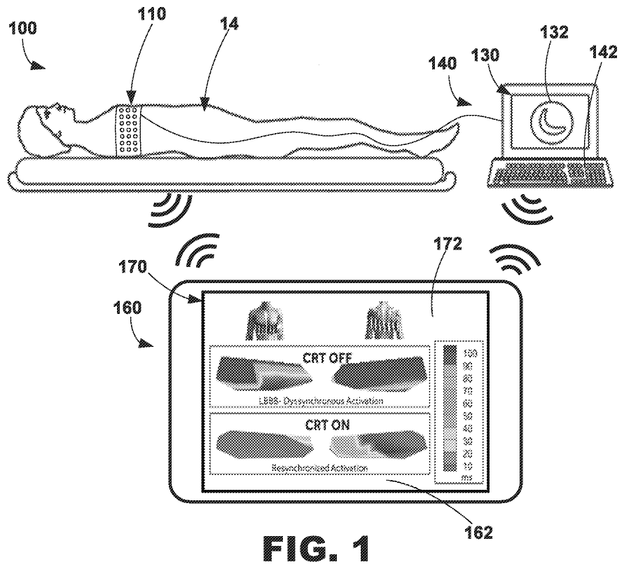

Illustrative systems and methods shall be described with reference to FIGS. 1-13. It will be apparent to one skilled in the art that elements or processes from one embodiment may be used in combination with elements or processes of the other embodiments, and that the possible embodiments of such systems, methods, and devices using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

A plurality of electrocardiogram (ECG) signals (e.g., torso-surface potentials) may be measured, or monitored, using a plurality of external electrodes positioned about the surface, or skin, of a patient. The ECG signals may be used to evaluate and configure cardiac therapy such as, e.g., cardiac therapy provide by an implantable medical device performing cardiac resynchronization therapy (CRT). As described herein, the ECG signals may be gathered or obtained noninvasively since, e.g., implantable electrodes may not be used to measure the ECG signals. Further, the ECG signals may be used to determine cardiac electrical activation times, which may be used to generate various metrics (e.g., electrical heterogeneity information) that may be used by a user (e.g., physician) to optimize one or more settings, or parameters, of cardiac therapy (e.g., pacing therapy) such as CRT.

Various illustrative systems, methods, and graphical user interfaces may be configured to use electrode apparatus including external electrodes, display apparatus, and computing apparatus to noninvasively assist a user (e.g., a physician) in the evaluation of cardiac health and/or the configuration (e.g., optimization) of cardiac therapy. An illustrative system 100 including electrode apparatus 110, computing apparatus 140, and a remote computing device 160 is depicted in FIG. 1.

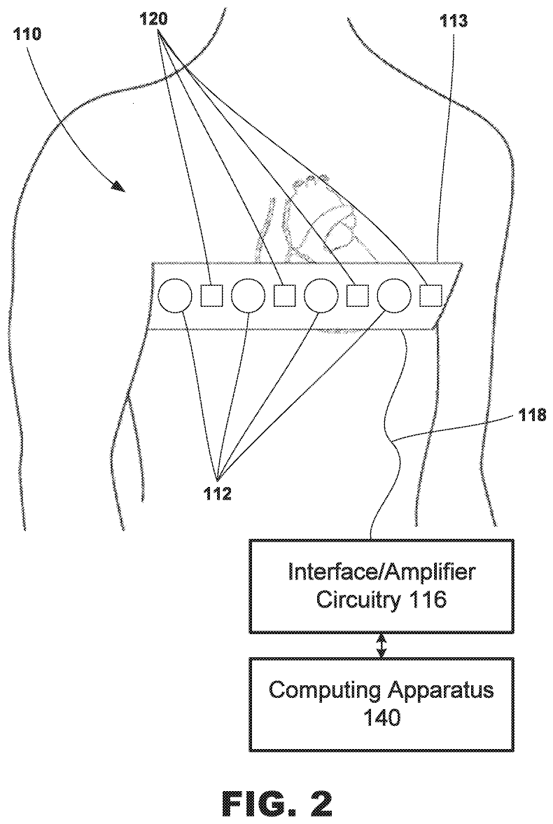

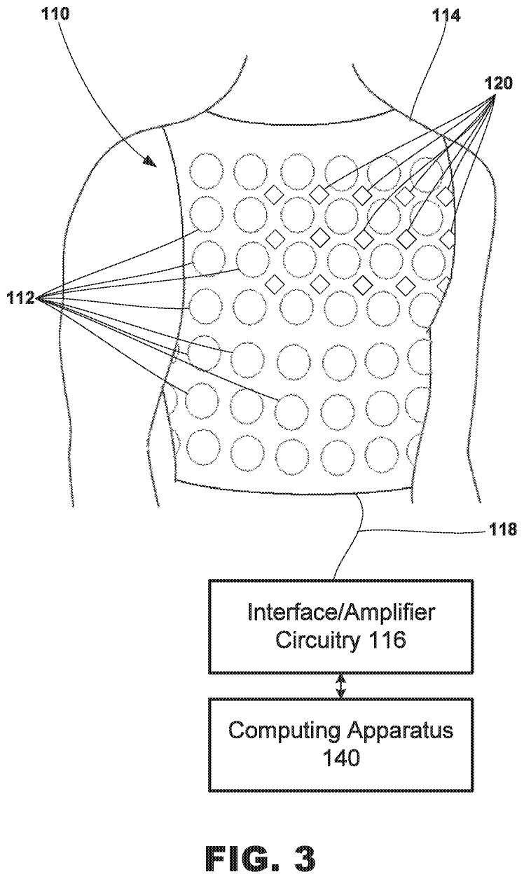

The electrode apparatus 110 as shown includes a plurality of electrodes incorporated, or included, within a band wrapped around the chest, or torso, of a patient 14. According to various embodiments, the electrode apparatus comprises about 40 electrodes. The electrode apparatus 110 is operatively coupled to the computing apparatus 140 (e.g., through one or wired electrical connections, wirelessly, etc.) to provide electrical signals from each of the electrodes to the computing apparatus 140 for analysis, evaluation, etc. Illustrative electrode apparatus may be described in U.S. Pat. No. 9,320,446 entitled “Bioelectric Sensor Device and Methods” filed Mar. 27, 2014 and issued on Mar. 26, 2016, which is incorporated herein by reference in its entirety. Further, illustrative electrode apparatus 110 will be described in more detail in reference to FIGS. 2-3.

Although not described herein, the illustrative system 100 may further include imaging apparatus. The imaging apparatus may be any type of imaging apparatus configured to image, or provide images of, at least a portion of the patient in a noninvasive manner. For example, the imaging apparatus may not use any components or parts that may be located within the patient to provide images of the patient except noninvasive tools such as contrast solution. It is to be understood that the illustrative systems, methods, and interfaces described herein may further use imaging apparatus to provide noninvasive assistance to a user (e.g., a physician) to locate, or place, one or more pacing electrodes proximate the patient's heart in conjunction with the configuration of cardiac therapy.

For example, the illustrative systems and methods may provide image guided navigation that may be used to navigate leads including electrodes, leadless electrodes, wireless electrodes, catheters, etc., within the patient's body while also providing noninvasive cardiac therapy configuration including determining an effective, or optimal, pre-excitation intervals such as A-V and V-V intervals, etc. Illustrative systems and methods that use imaging apparatus and/or electrode apparatus may be described in U.S. Pat. No. 9,877,789 to Ghosh, U.S. Pat. No. 10,251,555 to Ghosh et al., U.S. Pat. No. 9,924,884 to Ghosh et al., U.S. Pat. No. 10,064,567 to Ghosh et al., each of which is incorporated herein by reference in its entirety.

Illustrative imaging apparatus may be configured to capture x-ray images and/or any other alternative imaging modality. For example, the imaging apparatus may be configured to capture images, or image data, using isocentric fluoroscopy, bi-plane fluoroscopy, ultrasound, computed tomography (CT), multi-slice computed tomography (MSCT), magnetic resonance imaging (MRI), high frequency ultrasound (HIFU), optical coherence tomography (OCT), intra-vascular ultrasound (IVUS), two dimensional (2D) ultrasound, three dimensional (3D) ultrasound, four dimensional (4D) ultrasound, intraoperative CT, intraoperative MRI, etc. Further, it is to be understood that the imaging apparatus may be configured to capture a plurality of consecutive images (e.g., continuously) to provide video frame data. In other words, a plurality of images taken over time using the imaging apparatus may provide video frame, or motion picture, data. An exemplary system that employs ultrasound can be found in U.S. Pat. App. Pub. No. 2017/0303840 entitled NONINVASIVE ASSESSMENT OF CARDIAC RESYNCHRONIZATION THERAPY to Stadler et al., incorporated by reference in its entirety. Additionally, the images may also be obtained and displayed in two, three, or four dimensions. In more advanced forms, four-dimensional surface rendering of the heart or other regions of the body may also be achieved by incorporating heart data or other soft tissue data from a map or from pre-operative image data captured by MRI, CT, or echocardiography modalities. Image datasets from hybrid modalities, such as positron emission tomography (PET) combined with CT, or single photon emission computer tomography (SPECT) combined with CT, could also provide functional image data superimposed onto anatomical data, e.g., to be used to navigate implantable apparatus to target locations within the heart or other areas of interest.

Systems and/or imaging apparatus that may be used in conjunction with the illustrative systems and method described herein are described in U.S. Pat. App. Pub. No. 2005/0008210 to Evron et al. published on Jan. 13, 2005, U.S. Pat. App. Pub. No. 2006/0074285 to Zarkh et al. published on Apr. 6, 2006, U.S. Pat. No. 8,731,642 to Zarkh et al. issued on May 20, 2014, U.S. Pat. No. 8,861,830 to Brada et al. issued on Oct. 14, 2014, U.S. Pat. No. 6,980,675 to Evron et al. issued on Dec. 27, 2005, U.S. Pat. No. 7,286,866 to Okerlund et al. issued on Oct. 23, 2007, U.S. Pat. No. 7,308,297 to Reddy et al. issued on Dec. 11, 2011, U.S. Pat. No. 7,308,299 to Burrell et al. issued on Dec. 11, 2011, U.S. Pat. No. 7,321,677 to Evron et al. issued on Jan. 22, 2008, U.S. Pat. No. 7,346,381 to Okerlund et al. issued on Mar. 18, 2008, U.S. Pat. No. 7,454,248 to Burrell et al. issued on Nov. 18, 2008, U.S. Pat. No. 7,499,743 to Vass et al. issued on Mar. 3, 2009, U.S. Pat. No. 7,565,190 to Okerlund et al. issued on Jul. 21, 2009, U.S. Pat. No. 7,587,074 to Zarkh et al. issued on Sep. 8, 2009, U.S. Pat. No. 7,599,730 to Hunter et al. issued on Oct. 6, 2009, U.S. Pat. No. 7,613,500 to Vass et al. issued on Nov. 3, 2009, U.S. Pat. No. 7,742,629 to Zarkh et al. issued on Jun. 22, 2010, U.S. Pat. No. 7,747,047 to Okerlund et al. issued on Jun. 29, 2010, U.S. Pat. No. 7,778,685 to Evron et al. issued on Aug. 17, 2010, U.S. Pat. No. 7,778,686 to Vass et al. issued on Aug. 17, 2010, U.S. Pat. No. 7,813,785 to Okerlund et al. issued on Oct. 12, 2010, U.S. Pat. No. 7,996,063 to Vass et al. issued on Aug. 9, 2011, U.S. Pat. No. 8,060,185 to Hunter et al. issued on Nov. 15, 2011, and U.S. Pat. No. 8,401,616 to Verard et al. issued on Mar. 19, 2013, each of which is incorporated herein by reference in its entirety.

The computing apparatus 140 and the remote computing device 160 may each include display apparatus 130, 170, respectively, that may be configured to display and analyze data such as, e.g., electrical signals (e.g., electrocardiogram data), electrical activation times, electrical heterogeneity information, etc. For example, a plurality of electrical signals collected or monitored by the electrode apparatus 110 may be analyzed and evaluated by the computing apparatus 140 and the remote computing device 160 to determine or detect the QRS complexes therein such that, e.g., the QRS complexes can be further evaluated and analyzed for various metrics, activation time mapping, etc. Further, each QRS complex may be bracketed—the time period surrounding the QRS complex or the start and the end of the QRS complex may be determined. Further, for example, one cardiac cycle, or one heartbeat, of a plurality of cardiac cycles, or heartbeats, represented by the electrical signals collected or monitored by the electrode apparatus 110 may be analyzed and evaluated for one or more metrics including activation times and electrical heterogeneity information that may be pertinent to the therapeutic nature of one or more parameters related to cardiac therapy such as, e.g., pacing parameters, lead location, etc. More specifically, for example, the QRS complex of a single cardiac cycle may be evaluated for one or more metrics such as, e.g., QRS onset, QRS offset, QRS peak, electrical heterogeneity information (EHI), electrical activation times referenced to earliest activation time, left ventricular or thoracic standard deviation of electrical activation times (LVED), standard deviation of activation times (SDAT), average left ventricular or thoracic surrogate electrical activation times (LVAT), QRS duration (e.g., interval between QRS onset to QRS offset), difference between average left surrogate and average right surrogate activation times, relative or absolute QRS morphology, difference between a higher percentile and a lower percentile of activation times (higher percentile may be 90%, 80%, 75%, 70%, etc. and lower percentile may be 10%, 15%, 20%, 25% and 30%, etc.), other statistical measures of central tendency (e.g., median or mode), dispersion (e.g., mean deviation, standard deviation, variance, interquartile deviations, range), etc. Further, each of the one or more metrics may be location specific. For example, some metrics may be computed from signals recorded, or monitored, from electrodes positioned about a selected area of the patient such as, e.g., the left side of the patient, the right side of the patient, etc.

In at least one embodiment, activation maps may be created by interpolating a 2-by-20 matrix of activation times by first using an inverse distance-weighted interpolation step followed by a two-dimensional bi-cubic interpolation method. More specifically, the earliest (minimum) activation time across all valid electrodes may be determined and subtracted from each activation time. Over all electrodes, if an electrode is marked as valid, then the activation time is directly used in the bi-cubic interpolation step. If an electrode is marked as invalid, all valid electrodes are found within the same belt plane (anterior or posterior) and the contribution of each valid electrode to the interpolation is its activation time value weighted by the inverse of the distance squared from the invalid electrode. For instance,

$${AT}_{invalid} = \frac{{\sum }_{k = 1}^{N} {( \frac{1}{{dist}_{k}} )}^{2} \times {AT}_{k , valid}}{{\sum }_{k = 1}^{N} {( \frac{1}{{dist}_{k}} )}^{2}}$$

$$Where$$

$${dist}_{k} = \sqrt{{( {x}_{invalid} - {x}_{k , invalid} )}^{2} + {( {y}_{invalid} - {y}_{k , invalid} )}^{2}} .$$

Further, within the 2×10 array of activation times on each belt plane, for each set of 2×2 neighboring points that form a ‘unit square’, a system of 16 equations may be solved to find 16 coefficients of a two-dimensional polynomial function that can find the interpolated value at any fractional part within the unit square. Such process may be repeated for all possible neighboring 2×2 point sets.

In at least one embodiment, one or both of the computing apparatus 140 and the remote computing device 160 may be a server, a personal computer, a tablet computer, a mobile device, and a cellular telephone. The computing apparatus 140 may be configured to receive input from input apparatus 142 (e.g., a keyboard) and transmit output to the display apparatus 130, and the remote computing device 160 may be configured to receive input from input apparatus 162 (e.g., a touchscreen) and transmit output to the display apparatus 170. One or both of the computing apparatus 140 and the remote computing device 160 may include data storage that may allow for access to processing programs or routines and/or one or more other types of data, e.g., for analyzing a plurality of electrical signals captured by the electrode apparatus 110, for determining or detecting QRS complexes and time periods related thereto, for determining QRS onsets, QRS offsets, medians, modes, averages, peaks or maximum values, valleys or minimum values, for determining electrical activation times, for driving a graphical user interface configured to noninvasively assist a user in configuring one or more pacing parameters, or settings, such as, e.g., pacing rate, ventricular pacing rate, A-V interval, V-V interval, pacing pulse width, pacing vector, multipoint pacing vector (e.g., left ventricular vector quad lead), pacing voltage, pacing configuration (e.g., biventricular pacing, right ventricle only pacing, left ventricle only pacing, etc.), and arrhythmia detection and treatment, rate adaptive settings and performance, etc.

The computing apparatus 140 may be operatively coupled to the input apparatus 142 and the display apparatus 130 to, e.g., transmit data to and from each of the input apparatus 142 and the display apparatus 130, and the remote computing device 160 may be operatively coupled to the input apparatus 162 and the display apparatus 170 to, e.g., transmit data to and from each of the input apparatus 162 and the display apparatus 170. For example, the computing apparatus 140 and the remote computing device 160 may be electrically coupled to the input apparatus 142, 162 and the display apparatus 130, 170 using, e.g., analog electrical connections, digital electrical connections, wireless connections, bus-based connections, network-based connections, internet-based connections, etc. As described further herein, a user may provide input to the input apparatus 142, 162 to view and/or select one or more pieces of configuration information related to the cardiac therapy delivered by cardiac therapy apparatus such as, e.g., an implantable medical device.

Although as depicted the input apparatus 142 is a keyboard and the input apparatus 162 is a touchscreen, it is to be understood that the input apparatus 142, 162 may include any apparatus capable of providing input to the computing apparatus 140 and the computing device 160 to perform the functionality, methods, and/or logic described herein. For example, the input apparatus 142, 162 may include a keyboard, a mouse, a trackball, a touchscreen (e.g., capacitive touchscreen, a resistive touchscreen, a multi-touch touchscreen, etc.), etc. Likewise, the display apparatus 130, 170 may include any apparatus capable of displaying information to a user, such as a graphical user interface 132, 172 including electrode status information, graphical maps of electrical activation, a plurality of signals for the external electrodes over one or more heartbeats, QRS complexes, various cardiac therapy scenario selection regions, various rankings of cardiac therapy scenarios, various pacing parameters, electrical heterogeneity information (EHI), textual instructions, graphical depictions of anatomy of a human heart, images or graphical depictions of the patient's heart, graphical depictions of locations of one or more electrodes, graphical depictions of a human torso, images or graphical depictions of the patient's torso, graphical depictions or actual images of implanted electrodes and/or leads, etc. Further, the display apparatus 130, 170 may include a liquid crystal display, an organic light-emitting diode screen, a touchscreen, a cathode ray tube display, etc.

The processing programs or routines stored and/or executed by the computing apparatus 140 and the remote computing device 160 may include programs or routines for computational mathematics, matrix mathematics, decomposition algorithms, compression algorithms (e.g., data compression algorithms), calibration algorithms, image construction algorithms, signal processing algorithms (e.g., various filtering algorithms, Fourier transforms, fast Fourier transforms, etc.), standardization algorithms, comparison algorithms, vector mathematics, or any other processing used to implement one or more illustrative methods and/or processes described herein. Data stored and/or used by the computing apparatus 140 and the remote computing device 160 may include, for example, electrical signal/waveform data from the electrode apparatus 110 (e.g., a plurality of QRS complexes), electrical activation times from the electrode apparatus 110, cardiac sound/signal/waveform data from acoustic sensors, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein (e.g., electrical signals, electrical heterogeneity information, etc.), or any other data that may be used for carrying out the one and/or more processes or methods described herein.

In one or more embodiments, the illustrative systems, methods, and interfaces may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other devices and/or methods as described herein or as would be applied in a known fashion.

The one or more programs used to implement the systems, methods, and/or interfaces described herein may be provided using any programmable language, e.g., a high-level procedural and/or object orientated programming language that is suitable for communicating with a computer system. Any such programs may, for example, be stored on any suitable device, e.g., a storage media, that is readable by a general or special purpose program running on a computer system (e.g., including processing apparatus) for configuring and operating the computer system when the suitable device is read for performing the procedures described herein. In other words, at least in one embodiment, the illustrative systems, methods, and interfaces may be implemented using a computer readable storage medium, configured with a computer program, where the storage medium so configured causes the computer to operate in a specific and predefined manner to perform functions described herein. Further, in at least one embodiment, the illustrative systems, methods, and interfaces may be described as being implemented by logic (e.g., object code) encoded in one or more non-transitory media that includes code for execution and, when executed by a processor or processing circuitry, is operable to perform operations such as the methods, processes, and/or functionality described herein.

The computing apparatus 140 and the remote computing device 160 may be, for example, any fixed or mobile computer system (e.g., a controller, a microcontroller, a personal computer, minicomputer, tablet computer, etc.). The exact configurations of the computing apparatus 140 and the remote computing device 160 are not limiting, and essentially any device capable of providing suitable computing capabilities and control capabilities (e.g., signal analysis, mathematical functions such as medians, modes, averages, maximum value determination, minimum value determination, slope determination, minimum slope determination, maximum slope determination, graphics processing, etc.) may be used. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by the computing apparatus 140 and the remote computing device 160 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes, or programs (e.g., the functionality provided by such systems, processes, or programs) described herein.

The illustrative electrode apparatus 110 may be configured to measure body-surface potentials of a patient 14 and, more particularly, torso-surface potentials of a patient 14. As shown in FIG. 2, the illustrative electrode apparatus 110 may include a set, or array, of external electrodes 112, a strap 113, and interface/amplifier circuitry 116. The electrodes 112 may be attached, or coupled, to the strap 113 and the strap 113 may be configured to be wrapped around the torso of a patient 14 such that the electrodes 112 surround the patient's heart. As further illustrated, the electrodes 112 may be positioned around the circumference of a patient 14, including the posterior, lateral, posterolateral, anterolateral, and anterior locations of the torso of a patient 14.

The illustrative electrode apparatus 110 may be further configured to measure, or monitor, sounds from at least one or both the patient 14. As shown in FIG. 2, the illustrative electrode apparatus 110 may include a set, or array, of acoustic sensors 120 attached, or coupled, to the strap 113. The strap 113 may be configured to be wrapped around the torso of a patient 14 such that the acoustic sensors 120 surround the patient's heart. As further illustrated, the acoustic sensors 120 may be positioned around the circumference of a patient 14, including the posterior, lateral, posterolateral, anterolateral, and anterior locations of the torso of a patient 14.

Further, the electrodes 112 and the acoustic sensors 120 may be electrically connected to interface/amplifier circuitry 116 via wired connection 118. The interface/amplifier circuitry 116 may be configured to amplify the signals from the electrodes 112 and the acoustic sensors 120 and provide the signals to one or both of the computing apparatus 140 and the remote computing device 160. Other illustrative systems may use a wireless connection to transmit the signals sensed by electrodes 112 and the acoustic sensors 120 to the interface/amplifier circuitry 116 and, in turn, to one or both of the computing apparatus 140 and the remote computing device 160, e.g., as channels of data. In one or more embodiments, the interface/amplifier circuitry 116 may be electrically coupled to the computing apparatus 140 using, e.g., analog electrical connections, digital electrical connections, wireless connections, bus-based connections, network-based connections, internet-based connections, etc.

Although in the example of FIG. 2 the electrode apparatus 110 includes a strap 113, in other examples any of a variety of mechanisms, e.g., tape or adhesives, may be employed to aid in the spacing and placement of electrodes 112 and the acoustic sensors 120. In some examples, the strap 113 may include an elastic band, strip of tape, or cloth. Further, in some examples, the strap 113 may be part of, or integrated with, a piece of clothing such as, e.g., a t-shirt. In other examples, the electrodes 112 and the acoustic sensors 120 may be placed individually on the torso of a patient 14. Further, in other examples, one or both of the electrodes 112 (e.g., arranged in an array) and the acoustic sensors 120 (e.g., also arranged in an array) may be part of, or located within, patches, vests, and/or other manners of securing the electrodes 112 and the acoustic sensors 120 to the torso of the patient 14. Still further, in other examples, one or both of the electrodes 112 and the acoustic sensors 120 may be part of, or located within, two sections of material or two patches. One of the two patches may be located on the anterior side of the torso of the patient 14 (to, e.g., monitor electrical signals representative of the anterior side of the patient's heart, measure surrogate cardiac electrical activation times representative of the anterior side of the patient's heart, monitor or measure sounds of the anterior side of the patient, etc.) and the other patch may be located on the posterior side of the torso of the patient 14 (to, e.g., monitor electrical signals representative of the posterior side of the patient's heart, measure surrogate cardiac electrical activation times representative of the posterior side of the patient's heart, monitor or measure sounds of the posterior side of the patient, etc.). And still further, in other examples, one or both of the electrodes 112 and the acoustic sensors 120 may be arranged in a top row and bottom row that extend from the anterior side of the patient 14 across the left side of the patient 14 to the posterior side of the patient 14. Yet still further, in other examples, one or both of the electrodes 112 and the acoustic sensors 120 may be arranged in a curve around the armpit area and may have an electrode/sensor-density that less dense on the right thorax that the other remaining areas.

The electrodes 112 may be configured to surround the heart of the patient 14 and record, or monitor, the electrical signals associated with the depolarization and repolarization of the heart after the signals have propagated through the torso of a patient 14. Each of the electrodes 112 may be used in a unipolar configuration to sense the torso-surface potentials that reflect the cardiac signals. The interface/amplifier circuitry 116 may also be coupled to a return or indifferent electrode (not shown) that may be used in combination with each electrode 112 for unipolar sensing.

In some examples, the electrode apparatus 110 may include about 12 to about 50 electrodes 112 and about 12 to about 50 acoustic sensors 120 spatially distributed around the torso of a patient. Other configurations may have more or fewer electrodes 112 and more or fewer acoustic sensors 120. It is to be understood that the electrodes 112 and acoustic sensors 120 may not be arranged or distributed in an array extending all the way around or completely around the patient 14. Instead, the electrodes 112 and acoustic sensors 120 may be arranged in an array that extends only part of the way or partially around the patient 14. For example, the electrodes 112 and acoustic sensors 120 may be distributed on the anterior, posterior, and left sides of the patient with less or no electrodes and acoustic sensors proximate the right side (including posterior and anterior regions of the right side of the patient).

The computing apparatus 140 may record and analyze the torso-surface potential signals sensed by electrodes 112 and the sound signals sensed by the acoustic sensors 120, which are amplified/conditioned by the interface/amplifier circuitry 116. The computing apparatus 140 may be configured to analyze the electrical signals from the electrodes 112 to detect or determine QRS complexes, QRS onsets and offsets, and time periods related thereto and to provide electrocardiogram (ECG) signals, information, or data from the patient's heart as will be further described herein. The computing apparatus 140 may be configured to analyze the electrical signals from the acoustic sensors 120 to provide sound signals, information, or data from the patient's body and/or devices implanted therein (such as a left ventricular assist device).

Additionally, the computing apparatus 140 and the remote computing device 160 may be configured to provide graphical user interfaces 132, 172 depicting various information related to the electrode apparatus 110 and the data gathered, or sensed, using the electrode apparatus 110. For example, the graphical user interfaces 132, 172 may depict ECGs including QRS complexes obtained using the electrode apparatus 110 and sound data including sound waves obtained using the acoustic sensors 120 as well as other information related thereto. Illustrative systems and methods may noninvasively use the electrical information collected using the electrode apparatus 110 and the sound information collected using the acoustic sensors 120 to evaluate a patient's cardiac health and to evaluate and configure cardiac therapy being delivered to the patient.

Further, the electrode apparatus 110 may further include reference electrodes and/or drive electrodes to be, e.g. positioned about the lower torso of the patient 14, that may be further used by the system 100. For example, the electrode apparatus 110 may include three reference electrodes, and the signals from the three reference electrodes may be combined to provide a reference signal. Further, the electrode apparatus 110 may use of three caudal reference electrodes (e.g., instead of standard references used in a Wilson Central Terminal) to get a “true” unipolar signal with less noise from averaging three caudally located reference signals.

FIG. 3 illustrates another illustrative electrode apparatus 110 that includes a plurality of electrodes 112 configured to surround the heart of the patient 14 and record, or monitor, the electrical signals associated with the depolarization and repolarization of the heart after the signals have propagated through the torso of the patient 14 and a plurality of acoustic sensors 120 configured to surround the heart of the patient 14 and record, or monitor, the sound signals associated with the heart after the signals have propagated through the torso of the patient 14. The electrode apparatus 110 may include a vest 114 upon which the plurality of electrodes 112 and the plurality of acoustic sensors 120 may be attached, or to which the electrodes 112 and the acoustic sensors 120 may be coupled. In at least one embodiment, the plurality, or array, of electrodes 112 may be used to collect electrical information such as, e.g., surrogate electrical activation times. Similar to the electrode apparatus 110 of FIG. 2, the electrode apparatus 110 of FIG. 3 may include interface/amplifier circuitry 116 electrically coupled to each of the electrodes 112 and the acoustic sensors 120 through a wired connection 118 and be configured to transmit signals from the electrodes 112 and the acoustic sensors 120 to computing apparatus 140. As illustrated, the electrodes 112 and the acoustic sensors 120 may be distributed over the torso of a patient 14, including, for example, the posterior, lateral, posterolateral, anterolateral, and anterior locations of the torso of a patient 14.

The vest 114 may be formed of fabric with the electrodes 112 and the acoustic sensors 120 attached to the fabric. The vest 114 may be configured to maintain the position and spacing of electrodes 112 and the acoustic sensors 120 on the torso of the patient 14. Further, the vest 114 may be marked to assist in determining the location of the electrodes 112 and the acoustic sensors 120 on the surface of the torso of the patient 14. In some examples, there may be about 25 to about 256 electrodes 112 and about 25 to about 256 acoustic sensors 120 distributed around the torso of the patient 14, though other configurations may have more or fewer electrodes 112 and more or fewer acoustic sensors 120.

The illustrative systems, methods, and interfaces may be used to provide noninvasive assistance to a user in the evaluation of a patient's cardiac health (e.g., prior to or during delivery of cardiac therapy) and/or evaluation and configuration of cardiac therapy being presently delivered to the patient (e.g., by an implantable medical device delivering pacing therapy, by a LVAD, etc.). Further, it is to be understood that the computing apparatus 140 and the remote computing device 160 may be operatively coupled to each other in a plurality of different ways so as to perform, or execute, the functionality described herein. For example, in the embodiment depicted, the computing device 140 may be wireless operably coupled to the remote computing device 160 as depicted by the wireless signal lines emanating therebetween. Additionally, as opposed to wireless connections, one or more of the computing apparatus 140 and the remoting computing device 160 may be operably coupled through one or wired electrical connections.

According to embodiments described herein, the illustrative system 100, which may be referred to as an ECG belt system, may be used with cardiac therapy systems and devices (e.g., CRT pacing devices) to calculate various metrics related to the cardiac health of a patient (e.g., the standard deviation of activation times (SDAT)) across one or more cardiac cycles (or heart beats), and in particular, based on activation times or other data gathered during each QRS event of the cardiac cycle (heart beat). According to various embodiments, the illustrative system 100 may be used to calculate, or generate, electrical heterogeneity information such as, e.g., SDAT, of cardiac cycles during delivery of CRT (e.g., the SDAT for cardiac cycles where CRT paces are delivered). For example, the illustrative system 100 may be used to calculate electrical heterogeneity information for cardiac cycles during biventricular and/or left ventricular pacing. Further, embodiments described herein may be used to evaluate a patient's cardiac health and/or non-CRT pacing. If electrical heterogeneity information is inaccurate, the output of the illustrative system 100 could be misleading, which could potentially impact lead placement (e.g., an implantable lead not being placed at an optimal spot) and/or optimal device programming. For example, if the SDAT is inaccurate, the SDAT may be artificially low, which may cause a clinician to not relocate currently positioned lead as opposed to repositioning the lead to obtain a better response.

An illustrative process to determine SDAT may first calculate the mean of valid channels, or signals, as follows:

$${mean}_{AT} = \frac{{\sum }_{n = valid channels} {Activation\_time}_{n}}{Number\_of \_valid \_channels} .$$

Then, the squared standard deviation (for i=1 to number of valid channels) may be determined as follows:

$${squared}_{STD} = \frac{1}{N} * {( {Activation}_{{time}_{i}} - {mean}_{AT} )}^{2} .$$

Then, the SDAT may be calculated as follows:

$$SDAT = \sqrt{{squared}_{STD}} .$$

An illustrative process to determine LVAT may be described as the mean of the activation times of designated left ventricular channels, where each left ventricular activation time is referenced to the earliest activation time for all valid channels and may be represented as follows:

$$LVAT = \frac{{\sum }_{n = LV valid channels} {Activation\_time}_{n}}{Number\_of \_LV \_valid \_channels} .$$

In order to determine electrical heterogeneity information, each QRS event, or complex, with a plurality of cardiac cycles may be detected. Inaccurate detection of QRS events, or complexes, may cause false activation times to be detected leading to an inaccurate electrical heterogeneity information. According to various embodiments, a QRS event beginning or onset and/or a QRS event ending or offset is determined to give a bound on where to measure (e.g., determine a fiducial point within each of the plurality of cardiac signals). Embodiments herein describes a process for detecting QRS complexes and bounding them.

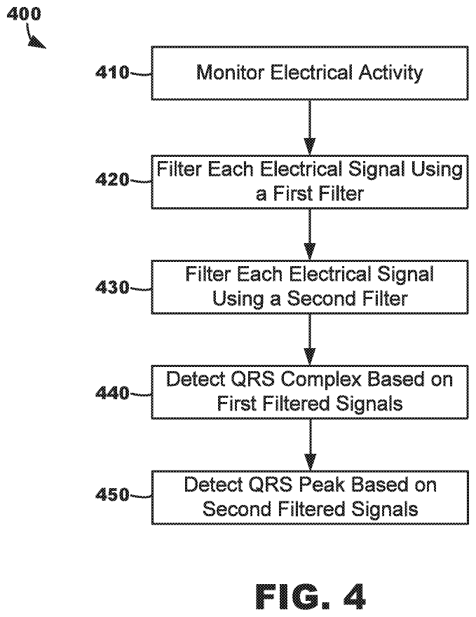

An exemplary method 400 for detecting QRS complexes is shown in FIG. 4 in accordance with embodiments described herein. Electrical activity from tissue of a patient is monitored 410 using a plurality of external electrodes to generate a plurality of electrical signals over time. The plurality of electrodes may be external surface electrodes configured in a band or a vest similar to as described herein with respect to FIGS. 1-3. Each of the electrodes may be positioned or located about the torso of the patient so as to monitor electrical activity (e.g., acquire torso-potentials) from a plurality of different locations about the torso of the patient. Each of the different locations where the electrodes are located may correspond to the electrical activation of different portions or regions of cardiac tissue of the patient's heart.

The plurality of electrical signals are filtered 420 using a first filter having a first frequency range to generate a plurality of first filtered signals. According to various configurations, the first filter is a bandpass filter configured to filter out signals outside of a range of about 0.05 Hz to about 150 Hz. In some cases, the first filter is a bandpass filter configured to filter out signals outside of a range of about 10 Hz to about 32 Hz.

The plurality of electrical signals are filtered 430 using a second filter having a second frequency range different than the first frequency range to generate a plurality of second filtered signals. According to various configurations, the second filter is a bandpass filter configured to filter out signals outside of a range of about 0.05 Hz to about 150 Hz. In some cases, the first filter is a bandpass filter configured to filter out signals outside of a range of about 0.5 Hz to about 20 Hz.

It may be described that the first and second filter provide zero-phase digital filtering (Bessel filtering) by processing the input data in both the forward and time reversed directions. This results in the following characteristics: zero-phase distortion; a filter transfer function that is equal to the squared magnitude of the original IIR filter derived from the Bessel filter with a bilinear transformation; and a filter order that is double the order of the filter coefficients of a designed infinite impulse response (IIR) filter. Further, the zero-phase filtering may be accomplished through the following steps: filter the data in the forward direction; time-reverse the data; filter the data in the time-reverse direction; and time-reverse the output of the previous step to obtain the original time correspondence of the data.

An illustrative IIR filter and coefficients is as follows:

$$y [ n ] = \frac{1}{{a}_{0}} ( {b}_{0} x [ n ] + {b}_{1} x [ n - 1 ] + {b}_{2} x [ n - 2 ] + {b}_{3} x [ n - 3 ] + {b}_{4} x [ n - 4 ] - {a}_{1} y [ n - 1 ] - {a}_{2} y [ n - 2 ] - {a}_{3} y [ n - 3 ] - {a}_{4} y [ n - 4 ] )$$

The coefficients for an illustrative 20 HZ low-pass IIR filter may be as follows: a0=1.000, a1=−1.790211747962553, a2=0.804402717060237, a3=0, a4=0, b0=0.003547742274421, b1=0.007095484548842, b2=0.003547742274420, b3=0, b4=0.

The coefficients for an illustrative 10-32 Hz bandpass IIR filter may be as follows: a 0=1, a1=−3.74704252837753, a2=5.28457739282789, a3=−3.32507118261580, a4=0.787677403869584, b0=0.00422287948064259, b1=−1.33226762955019e−15, b2=−0.00844575896128053, b3=−3.10862446895044e−15, b4=0.00422287948064337.

The coefficients for an illustrative 0.5-20 Hz bandpass IIR filter may be as follows: a0=1, a1=−3.79453631373899, a2=5.39795683527122, a3=−3.41222897663138, a4=0.808808595500041, b0=0.00338079909581213, b1=0, b2=−0.00676159819162336, b3=1.33226762955019e−15, b4=0.00338079909581224.

At least one QRS complex is detected 440 based on the plurality of first filtered signals. According to various configurations, detecting the at least one QRS complex comprises generating a first dispersion signal (e.g., standard deviation) based on the plurality of first filtered signals. The QRS complex is detected based on the dispersion signal. For example, the QRS complex may be detected by determining the peak of the first dispersion signal.

A QRS peak of the at least one QRS complex is detected 450 based on the plurality of second filtered signals and the detected at least one QRS complex. According to various configurations, detecting the at least one QRS peak comprises generating a second dispersion signal based on the plurality of second filtered signals. The second dispersion signal may be representative of the dispersion of the plurality of second filtered signals over time. The QRS peak is detected 450 based on the second dispersion signal.

The dispersion signals described herein may be representative of the dispersion of the plurality of cardiac signals over time. The dispersion signal may be the standard deviation of the plurality of cardiac signals over time. In other embodiments, the dispersion signals may be variance, a coefficient of variance, range, mean absolute deviation, a measure of central tendency (e.g., like the mean), interquartile deviation of amplitudes, median absolute deviation taken about a measure of central tendency like the mean, median, mode, and/or another statistical measure of the plurality of cardiac signals over time.

A blanking window of a predetermined length may be initialized based on the first dispersion signal. The blanking window may be used to determine a QRS peak by determining a maximum amplitude within the blanking window. According to various configurations, the predetermined length of the blanking window is in a range of about 100 ms to about 600 ms. In some cases, the predetermined length of the blanking window is about 200 ms. The predetermined length may be set in the factory and/or may be adjusted in the field. According to various implementations, the length of the blanking window is set based on at least one previously detected QRS complex.

In one or more embodiments a threshold function is be computed based on the QRS peak. The threshold function may be used to detect at least one subsequent QRS complex as will be described in more detail further herein. According to various implementations, the threshold function may be used to detect the at least one subsequent QRS complex by providing a sensitivity for the detection of the subsequent QRS complexes. The threshold function may be a decaying threshold signal that is based on one or both of the peaks of the first dispersion signal and the second dispersion signal. For example, decay parameters may be calculated using one or both of the peaks of the first dispersion signal and the second dispersion signal. The process may start over with each new QRS complex detected such that a different threshold function is computed for every QRS complex. According to various embodiments, the threshold function may be based on parameters of more than one previously detected QRS complex.

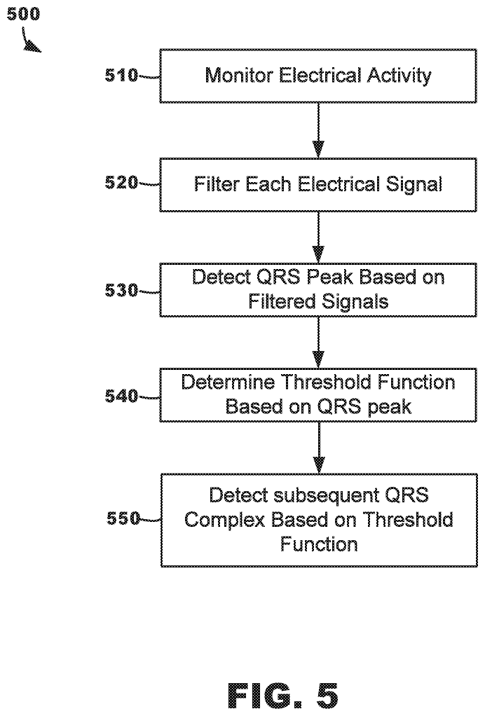

An exemplary method 500 for detecting a QRS complex and at least one subsequent QRS complex based on a threshold function is shown in FIG. 5. Electrical activity from tissue of a patient is monitored 510 using a plurality of external electrodes to generate a plurality of electrical signals over time. The plurality of electrical signals are filtered 520 using at least one filter to generate a plurality of filtered signals. According to various implementations, the plurality of electrical signals are filtered using one or both of the bandpass filters 420, 430 described in conjunction with FIG. 4. A QRS peak is detected 530 based on the plurality of filtered signals, and a threshold function may be determined 540 based on the QRS peak. At least one subsequent QRS complex may be detected 550 based on (e.g., using) the threshold function.

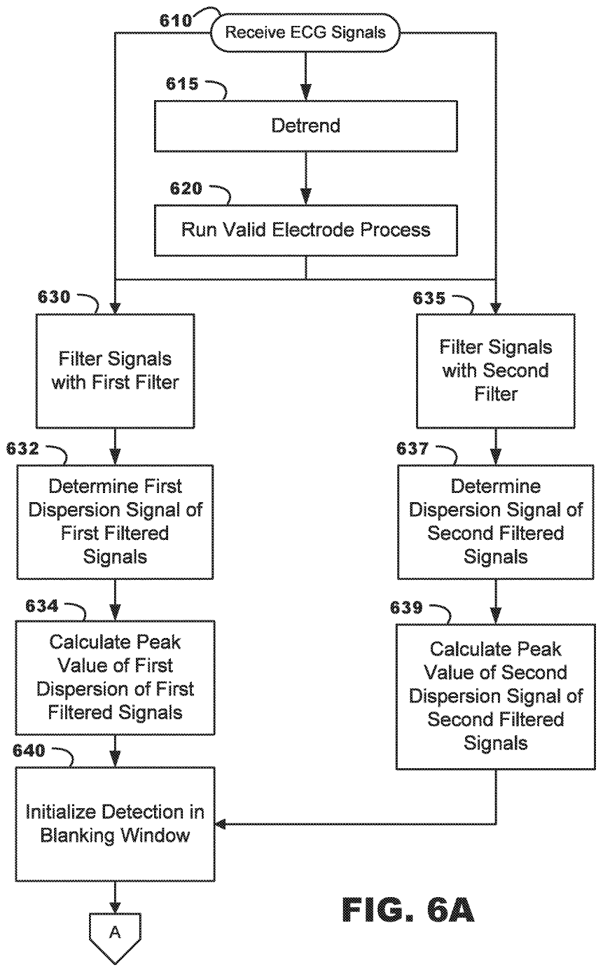

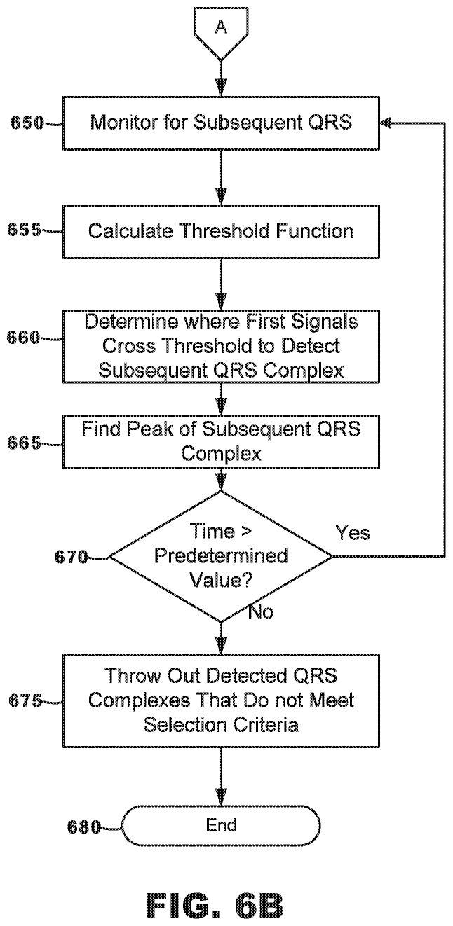

FIGS. 6A and 6B illustrate a more detailed process for detecting at least one QRS complex in accordance with embodiments described herein. A plurality of electrical signals, or ECG signals, received 610. According to various implementations, the ECG signals are received from an external electrode apparatus (e.g., ECG belt) as described herein. Initially, the ECG signals may be detrended 615 to remove any trend (e.g., a change in mean) over time from the ECG signals such as, e.g., mean increases or decreases over time. For example, it may be described that each ECG signal is de-trended to remove baseline drift using an algorithm based on the linear regression of the data. The algorithm may calculate the least squares regression line through all points within a 5000-sample recording, and then subtract the line from the original signal.

In at least one embodiment, the least squares regression line of the data may be defined as follows:

$${\hat{y}}_{i} = {b}_{0} + {b}_{1} {x}_{i}$$

using the following equations:

$${b}_{1} = \frac{{\sum }_{i = 1}^{n} ( {x}_{i} - \overline{x} ) ( {y}_{i} - \overline{y} )}{{\sum }_{i = 1}^{n} {( {x}_{i} - \overline{x} )}^{2}} and . {b}_{0} = \overline{y} - {b}_{1} \overline{x} .$$

Then, the predicted value may be subtracted from original data at each point in time to get the new de-trended value:

$${detrended}_{i} = {y}_{i} - {\hat{y}}_{i} .$$

Further, the system may determine 620 whether all of the electrodes are valid, which, for example, may involve determining if any electrodes are returning outlier data that could indicate that the electrodes are not receiving accurate data. Illustrative systems and methods for determining whether electrodes and/or ECG signals are valid may be described in U.S. Pat. No. 9,924,884 issued on Mar. 27, 2018, and U.S. Pat. No. 10,064,567 issued on Sep. 4, 2018, each of which are incorporated herein by reference in their entirety.

In at least one embodiment, determination of which ECG signals to include in the calculations is accomplished through the following steps, using the ECG signals filtered per the 20 Hz low-pass filter, and the portion of the signals defined by onset and offsets as described herein. Pearson correlation coefficients may be calculated for each electrode compared to its two neighbor electrodes. A signal may be declared invalid if at least one of the following is true: peak-to-peak>6 mV; peak-to-peak<(0.2*median peak-to-peak); peak-to-peak<=0.12 mV; correlation<=0.6 for both neighbors (does not apply to electrodes at either end, electrode 1 and electrode 40); absolute value of ratio of minimum amplitude relative to baseline and maximum amplitude relative to baseline is greater than 1, where baseline is the signal amplitude at onset AND minimum amplitude is at offset; and absolute value of ratio of maximum amplitude relative to baseline and minimum amplitude relative to baseline is greater than 1, where baseline is the signal amplitude at onset AND maximum amplitude is at offset; lead-off per the amplifier between onset and offset of the selected beat. Any signal that is not declared invalid may be determined to be valid.

The ECG signals are filtered 630 using a first filter to create first filtered signals. The first filter may the same filter as that described in conjunction with FIG. 4. A first dispersion signal is determined based on the of the first filtered signals 632. A peak value of the first dispersion signal is determined 634. According to various configurations, a minimum of a percentage of the peak and a predetermined value may be used to determine a location of the first QRS complex. For example, a minimum of about 35% of the peak and value about 0.075 may be used to determine a location of the first QRS complex. In some cases, it may be determined whether the 35% of the peak is less than a base threshold (e.g., 0.025). If it is determined that the minimum is less than the base threshold, the base threshold may be used to detect the time of the first QRS complex. The time of the first QRS complex may be used to initialize 640 a blanking window for use in determining the first QRS peak.

The ECG signals are filtered 635 with a second filter to create second filtered signals. The second filter may the same filter as that described in conjunction with FIG. 4. The ECG signals may be filtered 630 with the first filter in parallel as the ECG signals are filtered 635 by the second filter. In some cases, the ECG signals are filtered 630 with the first filter in series with the ECG signals being filtered 635 with the second filter. A second dispersion signal of the second filtered signals is determined 637. Detection of the first QRS peak is determined by determining 639 the maximum amplitude of the second dispersion signal within the blanking window.

The system may continue to monitor 650 the ECG signals for at least one subsequent QRS complex. The monitoring may initiate, or occur, after a predetermined amount of time has expired after the detected first QRS complex and/or a predetermined amount of time after the first QRS peak. For example, the system may begin to monitor for at least one subsequent QRS complex in a range of about 100 ms to about 600 ms after the detected first QRS peak. In some cases, the system begins to monitor for at least one subsequent QRS complex about 200 ms after the detected first QRS peak.

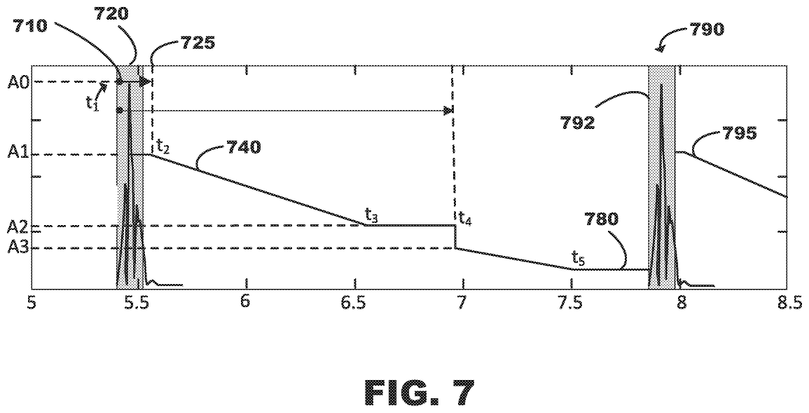

A threshold function may be calculated 655 based on the first detected QRS complex. The threshold function provides a sensitivity for detecting one or more subsequent QRS complexes. Additional detail with respected to an illustrative threshold function calculation or determination is described further herein with respect to FIG. 7. It is determined 660 where the second dispersion signal crosses the threshold based on the threshold function to detect a subsequent QRS complex. A new blanking window is initialized and the subsequent QRS peak is determined 665 using the same methods as those used for determining the peak of the first QRS peak.

It is determined 670 whether an elapsed time from the first QRS peak to a total monitoring time is less than a predetermined value. The predetermined value may be in a range of about 4000 ms to about 5000 ms. In some cases, the predetermined value is about 4750 ms. In some embodiments, the predetermined interval may be set based on the total monitoring time, e.g. total monitoring time—250 ms. If total monitoring time is 10 s (10000 ms) then value of this predetermined interval may be about 9750 ms. If it is determined that the elapsed time is less than the predetermined value, the system starts monitoring for another QRS complex. If it is determined 670 that the elapsed time is greater than the predetermined value, the system may continue to throw out 675 any detected QRS complexes that do not meet selection criteria and the process ends 680. The selection criteria may include that all detected QRS complexes are greater than a predetermined length. For example, the predetermined length may be in a range of about 250 ms to about 600 ms. In some cases, the predetermined length is about 400 ms. According to various implementations, the selection criteria may include that the QRS complexes be detected within a predetermined time limit. The predetermined time limit may be in a range of about 4000 ms to about 5000 ms. In some cases, the predetermined time limit is about 4750 ms.

FIG. 7 shows an example threshold function in accordance with embodiments described herein. Here, the maximum amplitude of the first filtered signal is used to detect a first QRS complex. According to various implementations, the maximum amplitude of the first filtered signal is used to determine the start 710 of the blanking window (A0) at time t₁. The maximum amplitude within the blanking window 720 is determined to be the QRS peak. After a predetermined amount of time 725 following the start 710 of the blanking window, the system may proceed to monitor for subsequent QRS complexes at time t₂.

A threshold function 740 may then be calculated based on the detected QRS peak. The threshold function may be configured to provide a sensitivity for detection of subsequent QRS complexes. According to various embodiments described herein, the threshold function 740 may be configured to decrease at time t₂ until at least one subsequent QRS complex is detected and/or a base threshold 780 is reached. According to various implementations, the base threshold is a base percentage of the QRS peak. For example, the base threshold may be in a range of about 0.5 percent to about 5 percent of the QRS peak. In some cases, the base threshold is about 3%

According to various implementations, the threshold function 740 is configured to decrease from a first threshold percentage (A1) at time t₂ to a second threshold percentage (A2) at time t₃. At least a portion of the decrease may be a substantially linear decrease or a nonlinear decrease. The threshold function may be configured to decrease from the A1 at time, t₂, (e.g., a predetermined amount of time after the start of the blanking window) to A2 at t₃. A1 may be in a range of about 50 percent to about 80 percent of the QRS peak 730. In some cases, A1 is about 60% of the QRS peak 730. A2 may be in a range of about 10 percent to about 50 percent of the QRS peak 730. In some cases, A2 is about 30% of the QRS peak.

The threshold function 730 may be configured to stay constant between t₃ and t₄. According to various configurations t1 is about 0 ms, t2 is about 200 ms, t3 is about 1200 ms, t4 is about 1700 ms and/or t5 is about 2200 ms. In some cases, the threshold function 740 is configured to drop at t₄ to a third threshold percentage (A3) of the QRS peak. A3 may be in a range of about 5 percent to about 20 percent of the QRS peak 730. In some cases, A3 is about 15% of the QRS peak. The threshold function may then be configured to drop linearly or nonlinearly between t₄ and t₅ to the base threshold percentage. If a subsequent QRS complex 790 is detected, a new blanking window 792 and a new threshold function 795 are established.

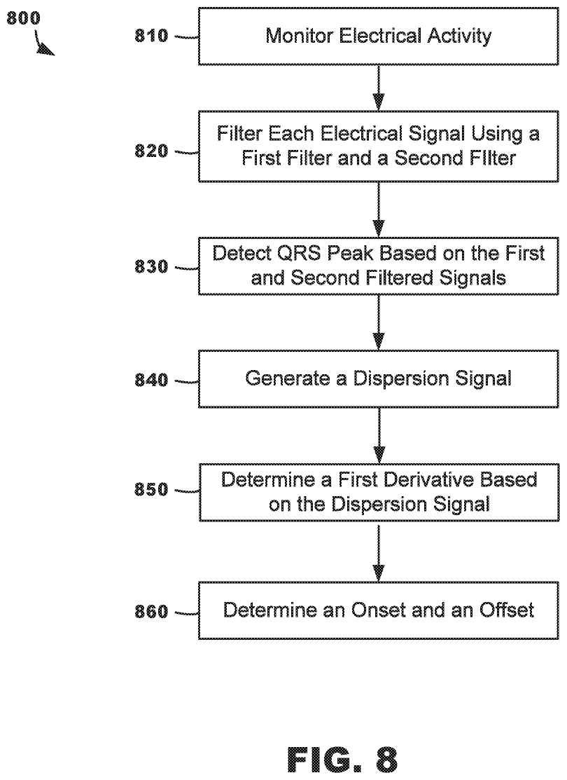

An exemplary method 800 for determining an onset and an offset of a corresponding QRS complex is shown in FIG. 8. Electrical activity from tissue of a patient is monitored 810 using a plurality of external electrodes to generate a plurality of electrical signals over time. The plurality of electrical signals are filtered 820 using a first filter and the second filter to generate a plurality of first filtered signals and plurality of second filtered signals, the first filter and the second filter having different frequency ranges. A QRS peak is detected 830 based on the plurality of first filtered signals and the plurality of second filtered signals as described herein with respect to FIGS. 4-6.

A dispersion signal may be generated 840 from the plurality of second filtered signals, and then a first derivative signal may be determined 850 based on the dispersion signal. At least one of a QRS onset time and a QRS offset time corresponding to the QRS complex may be determined 860 based on the first derivative signal.

According to various implementations, the onset time and the offset time may be used to calculate an activation time for the at least one QRS complex for each of the plurality of cardiac signals. In particular, a fiducial point (e.g., maximum negative slope) within a QRS duration between the QRS onset time value and the QRS offset time value may be determined, which may be used to determine an activation time. At least one metric of electrical heterogeneity may be determined based on the activation times of the plurality of cardiac signals for each QRS complex.

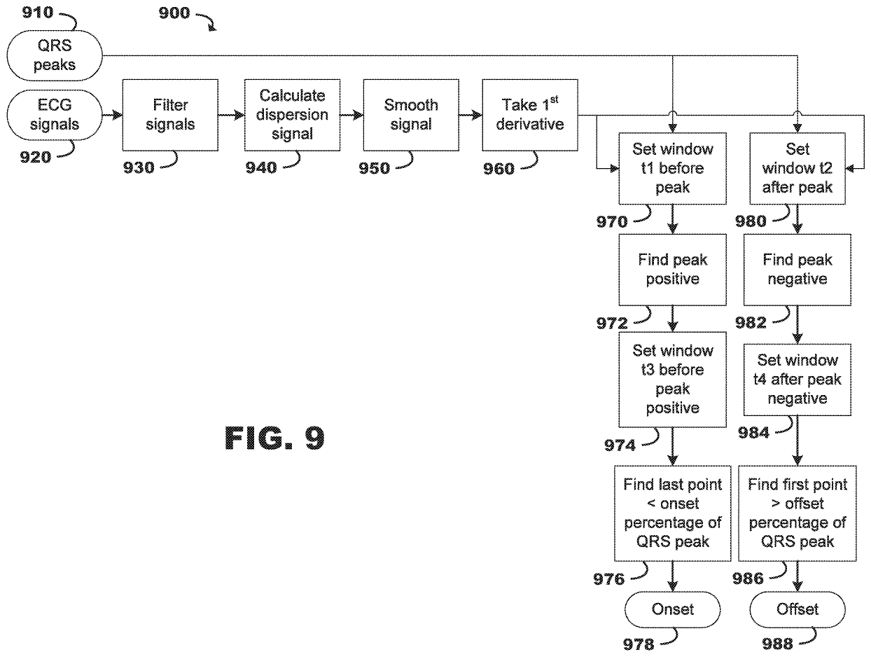

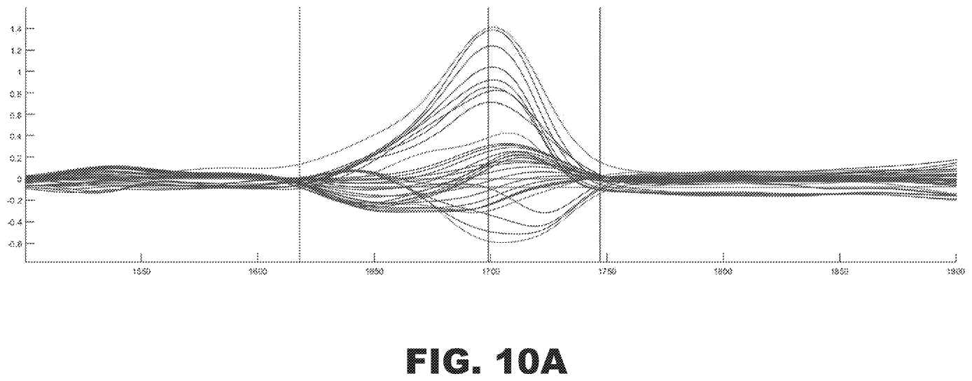

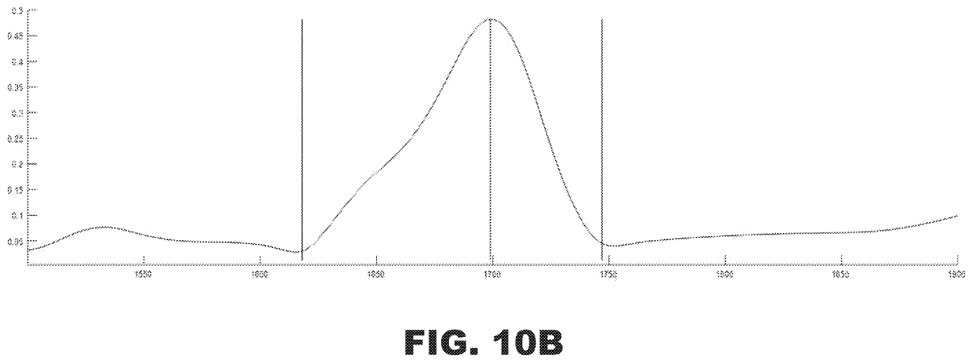

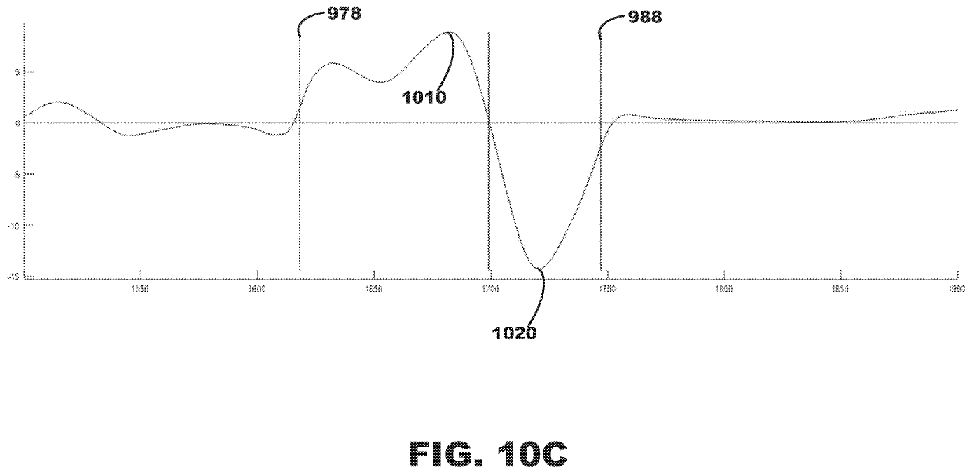

FIGS. 9 and 10A-10C illustrate a more detailed process 900 for determining the onset and offset values for at least one QRS complex in accordance with embodiments described herein. The timing of the one or more QRS peaks are received 910. In other words, the time at which each of the QRS peaks occurs may have been determined, e.g., using substantially the same processes that is described in conjunction with FIGS. 4-7 or may be determined using one or more different processes, and then received 910.

Additionally, the ECG signals may be received 920. For example, the ECG signals may be received from an ECG belt, and then the ECG signals are filtered 930 as shown in the example of FIG. 10A. According to various configurations, the ECG signals are filtered using one or more bandpass filters configured to remove, or “filter out,” signals outside of a predetermined range. For example, the bandpass filter may be configured to remove, or “filter out,” signals outside of a range of about 0.5 to about 20 Hz.

A dispersion signal of the filtered signals is determined 940 as shown in FIG. 10B, and then smoothed 950. The dispersion signal may be smoothed to reduce outlier data due to noise for example. According to various implementations, the dispersion signal may be smoothed using a 25-point equal-weighted smoother.

A first derivative signal of the smoothed signal is generated as shown in FIG. 10C. A first window is set 970 a predetermined amount of time before the QRS peak and is set 980 a predetermined amount of time after the QRS peak. The amount of time before the QRS peak and the amount of time after the QRS peak may be substantially the same. For example, the predetermined period of time before the QRS peak and after the QRS peak may be in a range of about 100 ms to about 150 ms. In some cases, the predetermined period of time before the QRS peak and after the QRS peak is about 120 ms. In one or more embodiments, the predetermined amount time before the QRS peak and the predetermined amount of time after the QRS peak are different. The predetermined amount before and after the QRS peak may be set in the factory and/or may be adjusted in the field.

The peak positive 1010 and the peak negative 1020 of the derivative signal is determined. A second window is set 974 a predetermined amount of time before the peak positive 1010 and is set 984 a predetermined amount of time after peak negative 1020. The amount of time before the peak positive and the amount of time after the peak negative may be substantially the same. For example, the predetermined amount of time before the peak positive and after the peak negative may be in a range of about 100 ms to about 150 ms. In some cases, predetermined amount of time before the peak positive and after the peak negative is about 120 ms. In some configurations, the predetermined amount of time before the peak positive and the predetermined amount of time after the peak negative are different than one another. The predetermined amount of time before the peak positive and after the peak negative may be set in the factory and/or may be adjusted in the field.

The QRS onset 978 is determined by finding 976 the last point in the second window that is less than a first predetermined percentage of the QRS peak. The first predetermined percentage may be in a range of about 10% to about 30% of the QRS peak. In some cases, the first predetermined percentage is about 20%.

The QRS offset 988 is determined by finding 986 a first time in the second window that is greater (i.e. more positive) than a second predetermined percentage of the QRS peak. The second predetermined percentage may be in a range of about 10% to about 30% of the QRS peak. In some cases, the second predetermined percentage is about 20%. It is to be understood that the first predetermined percentage and the second predetermined percentage may be set in the factory and/or may be programmable in the field. The first and the second predetermined percentage may be based on QRS history of a patient and/or multiple patients.

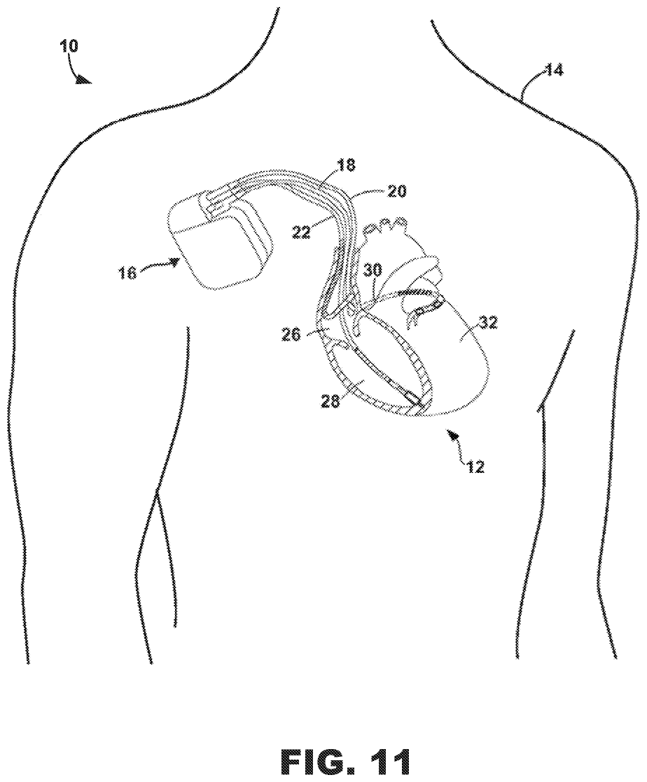

Illustrative cardiac therapy systems and devices may be further described herein with reference to FIGS. 11-13 that may utilizes the illustrative systems, interfaces, methods, and processes described herein with respect to FIGS. 1-10.

FIG. 11 is a conceptual diagram illustrating an illustrative therapy system 10 that may be used to deliver pacing therapy to a patient 14. Patient 14 may, but not necessarily, be a human. The therapy system 10 may include an implantable medical device 16 (IMD), which may be coupled to leads 18, 20, 22. The IMD 16 may be, e.g., an implantable pacemaker, cardioverter, and/or defibrillator, that delivers, or provides, electrical signals (e.g., paces, etc.) to and/or senses electrical signals from the heart 12 of the patient 14 via electrodes coupled to one or more of the leads 18, 20, 22.

The leads 18, 20, 22 extend into the heart 12 of the patient 14 to sense electrical activity of the heart 12 and/or to deliver electrical stimulation to the heart 12. In the example shown in FIG. 11, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, electrical signals attendant to the depolarization and repolarization of the heart 12 via electrodes coupled to at least one of the leads 18, 20, 22. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., A-V delay and other various timings, pulse wide, amplitude, voltage, burst length, etc. Further, the IMD 16 may be operable to use various electrode configurations to deliver pacing therapy, which may be unipolar, bipolar, quadripolar, or further multipolar. For example, a multipolar lead may include several electrodes that can be used for delivering pacing therapy. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the IMD 16 may detect arrhythmia of the heart 12, such as fibrillation of the ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of heart 12 is stopped.

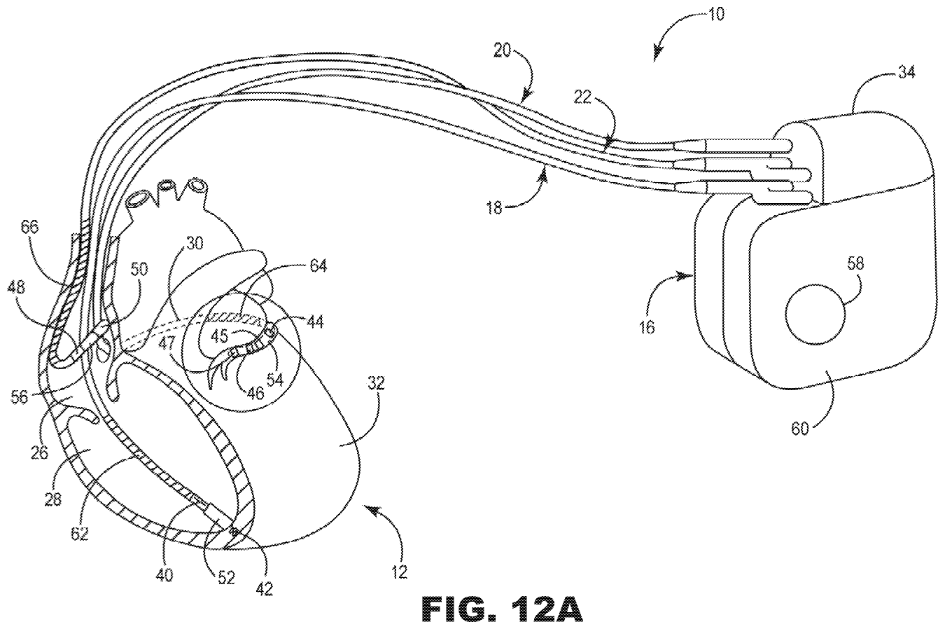

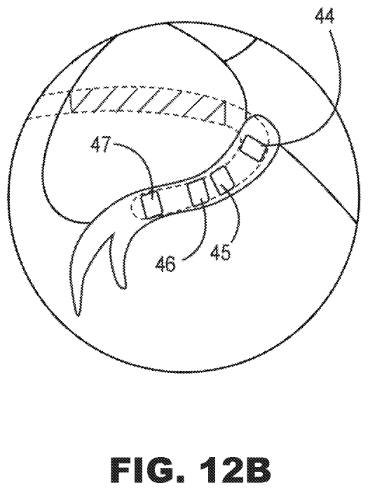

FIGS. 12A-12B are conceptual diagrams illustrating the IMD 16 and the leads 18, 20, 22 of therapy system 10 of FIG. 11 in more detail. The leads 18, 20, 22 may be electrically coupled to a therapy delivery module (e.g., for delivery of pacing therapy), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the IMD 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry a number of conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of ring electrodes, and the electrodes 42, 50 may take the form of extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

Additionally, electrodes 44, 45, 46 and 47 may have an electrode surface area of about 5.3 mm² to about 5.8 mm². Electrodes 44, 45, 46, and 47 may also be referred to as LV1, LV2, LV3, and LV4, respectively. The LV electrodes (i.e., left ventricle electrode 1 (LV1) 44, left ventricle electrode 2 (LV2) 45, left ventricle electrode 3 (LV3) 46, and left ventricle 4 (LV4) 47 etc.) on the lead 20 can be spaced apart at variable distances. For example, electrode 44 may be a distance of, e.g., about 21 millimeters (mm), away from electrode 45, electrodes 45 and 46 may be spaced a distance of, e.g. about 1.3 mm to about 1.5 mm, away from each other, and electrodes 46 and 47 may be spaced a distance of, e.g. 20 mm to about 21 mm, away from each other.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient's heart 12. In some examples, as illustrated in FIG. 12A, the IMD 16 includes one or more housing electrodes, such as housing electrode 58, which may be formed integrally with an outer surface of a housing 60 (e.g., hermetically-sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 12A, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient's heart during the delivery of pacing therapy (e.g., in combination with the housing electrode 58, or defibrillation electrode-to-housing electrode vector).

The configuration of the illustrative therapy system 10 illustrated in FIGS. 11-13 is merely one example. In other examples, the therapy system may include epicardial leads and/or patch electrodes instead of or in addition to the transvenous leads 18, 20, 22 illustrated in FIG. 11. Additionally, in other examples, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 11). Further, in one or more embodiments, the IMD 16 need not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

In other examples of therapy systems that provide electrical stimulation therapy to the heart 12, such therapy systems may include any suitable number of leads coupled to the IMD 16, and each of the leads may extend to any location within or proximate to the heart 12. For example, other examples of therapy systems may include three transvenous leads located as illustrated in FIGS. 11-13. Still further, other therapy systems may include a single lead that extends from the IMD 16 into the right atrium 26 or the right ventricle 28, or two leads that extend into a respective one of the right atrium 26 and the right ventricle 28.

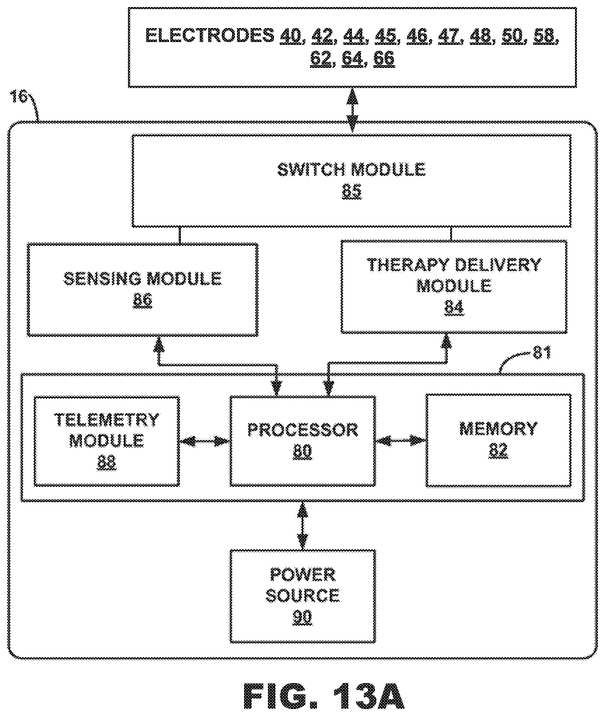

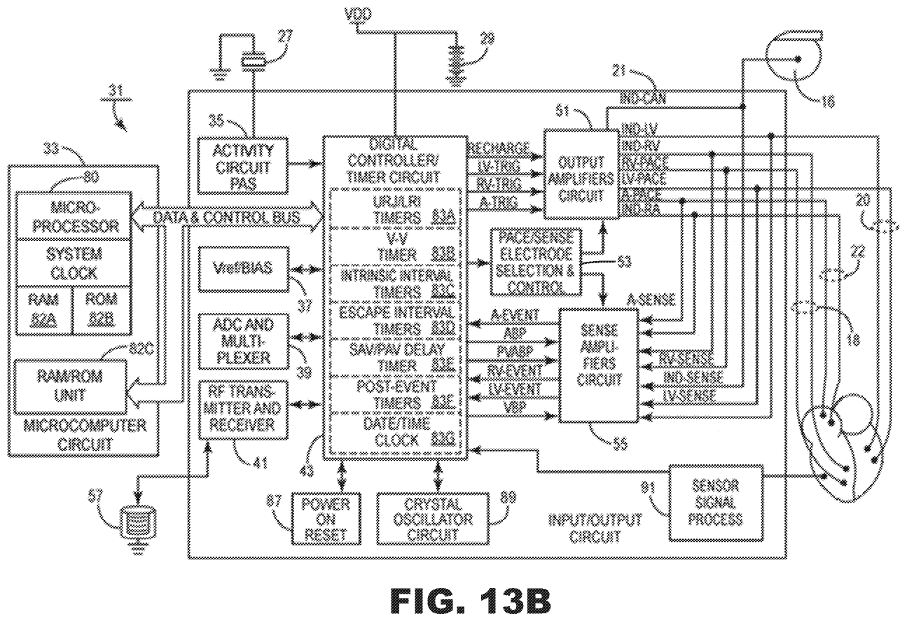

FIG. 13A is a functional block diagram of one illustrative configuration of the IMD 16. As shown, the IMD 16 may include a control module 81, a therapy delivery module 84 (e.g., which may include a stimulation generator), a sensing module 86, and a power source 90.

The control module, or apparatus, 81 may include a processor 80, memory 82, and a telemetry module, or apparatus, 88. The memory 82 may include computer-readable instructions that, when executed, e.g., by the processor 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically-erasable programmable ROM (EEPROM), flash memory, and/or any other digital media. An illustrative capture management module may be the left ventricular capture management (LVCM) module described in U.S. Pat. No. 7,684,863 entitled “LV THRESHOLD MEASUREMENT AND CAPTURE MANAGEMENT” and issued Mar. 23, 2010, which is incorporated herein by reference in its entirety.

The processor 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the processor 80 may include multiple components, such as any combination of one or more microprocessors, one or more controllers, one or more DSPs, one or more ASICs, and/or one or more FPGAs, as well as other discrete or integrated logic circuitry. The functions attributed to the processor 80 herein may be embodied as software, firmware, hardware, or any combination thereof.

The control module 81 may control the therapy delivery module 84 to deliver therapy (e.g., electrical stimulation therapy such as pacing) to the heart 12 according to a selected one or more therapy programs, which may be stored in the memory 82. More, specifically, the control module 81 (e.g., the processor 80) may control various parameters of the electrical stimulus delivered by the therapy delivery module 84 such as, e.g., A-V delays, V-V delays, pacing pulses with the amplitudes, pulse widths, frequency, or electrode polarities, etc., which may be specified by one or more selected therapy programs (e.g., A-V and/or V-V delay adjustment programs, pacing therapy programs, pacing recovery programs, capture management programs, etc.). As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured deliver one or more of these types of stimulation in the form of other signals, such as sine waves, square waves, and/or other substantially continuous time signals.

The IMD 16 may further include a switch module 85 and the control module 81 (e.g., the processor 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switching module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave alternans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 40, 42, 44, 45, 46, 47, 48, 50, 58, 62, 64, 66), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any necessary mathematical calculations may be performed by the processor 80 and any updating of the values or intervals controlled by the pacer timing and control module may take place following such interrupts. A portion of memory 82 may be configured as a plurality of recirculating buffers, capable of holding one or more series of measured intervals, which may be analyzed by, e.g., the processor 80 in response to the occurrence of a pace or sense interrupt to determine whether the patient's heart 12 is presently exhibiting atrial or ventricular tachyarrhythmia.

The telemetry module 88 of the control module 81 may include any suitable hardware, firmware, software, or any combination thereof for communicating with another device, such as a programmer. For example, under the control of the processor 80, the telemetry module 88 may receive downlink telemetry from and send uplink telemetry to a programmer with the aid of an antenna, which may be internal and/or external. The processor 80 may provide the data to be uplinked to a programmer and the control signals for the telemetry circuit within the telemetry module 88, e.g., via an address/data bus. In some examples, the telemetry module 88 may provide received data to the processor 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

FIG. 13B is another embodiment of a functional block diagram for IMD 16 that depicts bipolar RA lead 22, bipolar RV lead 18, and bipolar LV CS lead 20 without the LA CS pace/sense electrodes and coupled with an implantable pulse generator (IPG) circuit 31 having programmable modes and parameters of a bi-ventricular DDD/R type known in the pacing art. In turn, the sensor signal processing circuit 91 indirectly couples to the timing circuit 43 and via data and control bus to microcomputer circuitry 33. The IPG circuit 31 is illustrated in a functional block diagram divided generally into a microcomputer circuit 33 and a pacing circuit 21. The pacing circuit 21 includes the digital controller/timer circuit 43, the output amplifiers circuit 51, the sense amplifiers circuit 55, the RF telemetry transceiver 41, the activity sensor circuit 35 as well as a number of other circuits and components described below.

Crystal oscillator circuit 89 provides the basic timing clock for the pacing circuit 21 while battery 29 provides power. Power-on-reset circuit 87 responds to initial connection of the circuit to the battery for defining an initial operating condition and similarly, resets the operative state of the device in response to detection of a low battery condition. Reference mode circuit 37 generates stable voltage reference and currents for the analog circuits within the pacing circuit 21. Analog-to-digital converter (ADC) and multiplexer circuit 39 digitize analog signals and voltage to provide, e.g., real time telemetry of cardiac signals from sense amplifiers 55 for uplink transmission via RF transmitter and receiver circuit 41. Voltage reference and bias circuit 37, ADC and multiplexer 39, power-on-reset circuit 87, and crystal oscillator circuit 89 may correspond to any of those used in illustrative implantable cardiac pacemakers.

If the IPG is programmed to a rate responsive mode, the signals output by one or more physiologic sensors are employed as a rate control parameter (RCP) to derive a physiologic escape interval. For example, the escape interval is adjusted proportionally to the patient's activity level developed in the patient activity sensor (PAS) circuit 35 in the depicted, illustrative IPG circuit 31. The patient activity sensor 27 is coupled to the IPG housing and may take the form of a piezoelectric crystal transducer. The output signal of the patient activity sensor 27 may be processed and used as an RCP. Sensor 27 generates electrical signals in response to sensed physical activity that are processed by activity circuit 35 and provided to digital controller/timer circuit 43. Activity circuit 35 and associated sensor 27 may correspond to the circuitry disclosed in U.S. Pat. No. 5,052,388 entitled “METHOD AND APPARATUS FOR IMPLEMENTING ACTIVITY SENSING IN A PULSE GENERATOR” and issued on Oct. 1, 1991 and U.S. Pat. No. 4,428,378 entitled “RATE ADAPTIVE PACER” and issued on Jan. 31, 1984, each of which is incorporated herein by reference in its entirety. Similarly, the illustrative systems, apparatus, and methods described herein may be practiced in conjunction with alternate types of sensors such as oxygenation sensors, pressure sensors, pH sensors, and respiration sensors, for use in providing rate responsive pacing capabilities. Alternately, QT time may be used as a rate indicating parameter, in which case no extra sensor is required. Similarly, the illustrative embodiments described herein may also be practiced in non-rate responsive pacemakers.

Data transmission to and from the external programmer is accomplished by way of the telemetry antenna 57 and an associated RF transceiver 41, which serves both to demodulate received downlink telemetry and to transmit uplink telemetry. Uplink telemetry capabilities may include the ability to transmit stored digital information, e.g., operating modes and parameters, EGM histograms, and other events, as well as real time EGMs of atrial and/or ventricular electrical activity and marker channel pulses indicating the occurrence of sensed and paced depolarizations in the atrium and ventricle.

Microcomputer 33 contains a microprocessor 80 and associated system clock and on-processor RAM and ROM chips 82A and 82B, respectively. In addition, microcomputer circuit 33 includes a separate RAM/ROM chip 82C to provide additional memory capacity. Microprocessor 80 normally operates in a reduced power consumption mode and is interrupt driven. Microprocessor 80 is awakened in response to defined interrupt events, which may include A-TRIG, RV-TRIG, LV-TRIG signals generated by timers in digital timer/controller circuit 43 and A-EVENT, RV-EVENT, and LV-EVENT signals generated by sense amplifiers circuit 55, among others. The specific values of the intervals and delays timed out by digital controller/timer circuit 43 are controlled by the microcomputer circuit 33 by way of data and control bus from programmed-in parameter values and operating modes. In addition, if programmed to operate as a rate responsive pacemaker, a timed interrupt, e.g., every cycle or every two seconds, may be provided in order to allow the microprocessor to analyze the activity sensor data and update the basic A-A, V-A, or V-V escape interval, as applicable. In addition, the microprocessor 80 may also serve to define variable, operative A-V delay intervals, V-V delay intervals, and the energy delivered to each ventricle and/or atrium.

In one embodiment, microprocessor 80 is a custom microprocessor adapted to fetch and execute instructions stored in RAM/ROM unit 82 in a conventional manner. It is contemplated, however, that other implementations may be suitable to practice the present disclosure. For example, an off-the-shelf, commercially available microprocessor or microcontroller, or custom application-specific, hardwired logic, or state-machine type circuit may perform the functions of microprocessor 80.

Digital controller/timer circuit 43 operates under the general control of the microcomputer 33 to control timing and other functions within the pacing circuit 21 and includes a set of timing and associated logic circuits of which certain ones pertinent to the present disclosure are depicted. The depicted timing circuits include URI/LRI timers 83A, V-V delay timer 83B, intrinsic interval timers 83C for timing elapsed V-EVENT to V-EVENT intervals or V-EVENT to A-EVENT intervals or the V-V conduction interval, escape interval timers 83D for timing A-A, V-A, and/or V-V pacing escape intervals, an A-V delay interval timer 83E for timing the A-LVp delay (or A-RVp delay) from a preceding A-EVENT or A-TRIG, a post-ventricular timer 83F for timing post-ventricular time periods, and a date/time clock 83G.

The A-V delay interval timer 83E is loaded with an appropriate delay interval for one ventricular chamber (e.g., either an A-RVp delay or an A-LVp) to time-out starting from a preceding A-PACE or A-EVENT. The interval timer 83E triggers pacing stimulus delivery and can be based on one or more prior cardiac cycles (or from a data set empirically derived for a given patient).

The post-event timer 83F times out the post-ventricular time period following an RV-EVENT or LV-EVENT or a RV-TRIG or LV-TRIG and post-atrial time periods following an A-EVENT or A-TRIG. The durations of the post-event time periods may also be selected as programmable parameters stored in the microcomputer 33. The post-ventricular time periods include the PVARP, a post-atrial ventricular blanking period (PAVBP), a ventricular blanking period (VBP), a post-ventricular atrial blanking period (PVARP) and a ventricular refractory period (VRP) although other periods can be suitably defined depending, at least in part, on the operative circuitry employed in the pacing engine. The post-atrial time periods include an atrial refractory period (ARP) during which an A-EVENT is ignored for the purpose of resetting any A-V delay, and an atrial blanking period (ABP) during which atrial sensing is disabled. It should be noted that the starting of the post-atrial time periods and the A-V delays can be commenced substantially simultaneously with the start or end of each A-EVENT or A-TRIG or, in the latter case, upon the end of the A-PACE which may follow the A-TRIG. Similarly, the starting of the post-ventricular time periods and the V-A escape interval can be commenced substantially simultaneously with the start or end of the V-EVENT or V-TRIG or, in the latter case, upon the end of the V-PACE which may follow the V-TRIG. The microprocessor 80 also optionally calculates A-V delays, V-V delays, post-ventricular time periods, and post-atrial time periods that vary with the sensor-based escape interval established in response to the RCP(s) and/or with the intrinsic atrial and/or ventricular rate.

The output amplifiers circuit 51 contains a RA pace pulse generator (and a LA pace pulse generator if LA pacing is provided), a RV pace pulse generator, a LV pace pulse generator, and/or any other pulse generator configured to provide atrial and ventricular pacing. In order to trigger generation of an RV-PACE or LV-PACE pulse, digital controller/timer circuit 43 generates the RV-TRIG signal at the time-out of the A-RVp delay (in the case of RV pre-excitation) or the LV-TRIG at the time-out of the A-LVp delay (in the case of LV pre-excitation) provided by A-V delay interval timer 83E (or the V-V delay timer 83B). Similarly, digital controller/timer circuit 43 generates an RA-TRIG signal that triggers output of an RA-PACE pulse (or an LA-TRIG signal that triggers output of an LA-PACE pulse, if provided) at the end of the V-A escape interval timed by escape interval timers 83D.

The output amplifiers circuit 51 includes switching circuits for coupling selected pace electrode pairs from among the lead conductors and the IND-CAN electrode 20 to the RA pace pulse generator (and LA pace pulse generator if provided), RV pace pulse generator and LV pace pulse generator. Pace/sense electrode pair selection and control circuit 53 selects lead conductors and associated pace electrode pairs to be coupled with the atrial and ventricular output amplifiers within output amplifiers circuit 51 for accomplishing RA, LA, RV and LV pacing.

The sense amplifiers circuit 55 contains sense amplifiers for atrial and ventricular pacing and sensing. High impedance P-wave and R-wave sense amplifiers may be used to amplify a voltage difference signal that is generated across the sense electrode pairs by the passage of cardiac depolarization wavefronts. The high impedance sense amplifiers use high gain to amplify the low amplitude signals and rely on pass band filters, time domain filtering and amplitude threshold comparison to discriminate a P-wave or R-wave from background electrical noise. Digital controller/timer circuit 43 controls sensitivity settings of the atrial and ventricular sense amplifiers 55.

The sense amplifiers may be uncoupled from the sense electrodes during the blanking periods before, during, and after delivery of a pace pulse to any of the pace electrodes of the pacing system to avoid saturation of the sense amplifiers. The sense amplifiers circuit 55 includes blanking circuits for uncoupling the selected pairs of the lead conductors and the IND-CAN electrode 20 from the inputs of the RA sense amplifier (and LA sense amplifier if provided), RV sense amplifier and LV sense amplifier during the ABP, PVABP and VBP. The sense amplifiers circuit 55 also includes switching circuits for coupling selected sense electrode lead conductors and the IND-CAN electrode 20 to the RA sense amplifier (and LA sense amplifier if provided), RV sense amplifier and LV sense amplifier. Again, sense electrode selection and control circuit 53 selects conductors and associated sense electrode pairs to be coupled with the atrial and ventricular sense amplifiers within the output amplifiers circuit 51 and sense amplifiers circuit 55 for accomplishing RA, LA, RV, and LV sensing along desired unipolar and bipolar sensing vectors.

Right atrial depolarizations or P-waves in the RA-SENSE signal that are sensed by the RA sense amplifier result in a RA-EVENT signal that is communicated to the digital controller/timer circuit 43. Similarly, left atrial depolarizations or P-waves in the LA-SENSE signal that are sensed by the LA sense amplifier, if provided, result in a LA-EVENT signal that is communicated to the digital controller/timer circuit 43. Ventricular depolarizations or R-waves in the RV-SENSE signal are sensed by a ventricular sense amplifier result in an RV-EVENT signal that is communicated to the digital controller/timer circuit 43. Similarly, ventricular depolarizations or R-waves in the LV-SENSE signal are sensed by a ventricular sense amplifier result in an LV-EVENT signal that is communicated to the digital controller/timer circuit 43. The RV-EVENT, LV-EVENT, and RA-EVENT, LA-SENSE signals may be refractory or non-refractory and can inadvertently be triggered by electrical noise signals or aberrantly conducted depolarization waves rather than true R-waves or P-waves.

The techniques described in this disclosure, including those attributed to the IMD 16, the computing apparatus 140, and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

### ILLUSTRATIVE EMBODIMENTS

Embodiment 1. A system for use in cardiac evaluation comprising:
an electrode apparatus comprising a plurality of external electrodes to be disposed proximate a patient's skin; and
a computing apparatus comprising processing circuitry, the computing apparatus operably coupled to the electrode apparatus and configured to:
monitor electrical activity from tissue of a patient using a plurality of external electrodes to generate a plurality of electrical signals over time;
filter the plurality of electrical signals using a first filter having a first frequency range to generate a plurality of first filtered signals;
filter the plurality of electrical signals using a second filter having a second frequency range different than the first frequency range to generate a plurality of second filtered signals;
detect at least one QRS complex based on the plurality of first filtered signals; and
detect a QRS peak of the at least one QRS complex based on the plurality of second filtered signals and the detected at least one QRS complex.

Embodiment 2. The system of embodiment 1, wherein the first filter is a bandpass filter configured to filter out frequencies outside of a range of about 10 Hz to about 32 Hz.

Embodiment 3. The system as in any one of embodiments 1-2, wherein the second filter is a bandpass filter configured to filter out frequencies outside of a range of about 0.5 Hz to about 20 Hz.

Embodiment 4. The system as in any one of embodiments 1-3, wherein detecting the at least one QRS complex comprises:
generating a dispersion signal based on the plurality of first filtered signals, wherein the dispersion signal is representative of the dispersion of the plurality second filtered signals over time; and
detecting the at least one QRS complex based on the dispersion signal.

Embodiment 5. The system as in any one of embodiments 1-4, wherein detecting the QRS peak comprises:
determining a standard deviation of the plurality of second filtered signals;
initializing a blanking window of a predetermined length based standard deviation; and
detecting the peak amplitude of the plurality of second filtered signals within the blanking window.

Embodiment 6. The system as in any one of embodiments 1-5, wherein the computing device is further configured to:
determine a threshold function based on the QRS peak, the threshold function configured to provide sensitivity for detecting at least one subsequent QRS complex; and
detect the at least one subsequent QRS complex based on the threshold function.

Embodiment 7. The system as in any one of embodiments 1-6, wherein the computing apparatus is further configured to determine a QRS onset time value and a QRS offset time value corresponding to the at least one QRS complex.

Embodiment 8. The system of embodiment 7, wherein determining the QRS onset time value and the QRS offset time value comprises:
generating a dispersion signal from the plurality second filtered signals, wherein the dispersion signal is representative of the dispersion of the plurality second filtered signals over time,
determining a first derivative signal based on the dispersion signal;
determining a first window of the first derivative signal within a first time period before a peak positive value of the first derivative signal;
determining a second window of the first derivative signal within a second time period after a peak negative value of the first derivative signal;
determining the onset time value by determining a last point within the first window that is less than a first threshold; and
determining the offset time value by determining a first point within the second window that is greater than a second threshold.

Embodiment 9. The system of embodiment 7, wherein the computing apparatus is further configured to determine a plurality of activation times for the at least one QRS complex based on the plurality of cardiac signals within a QRS duration between the QRS onset time value and the QRS offset time value.

Embodiment 10. The system of embodiment 9, wherein the computing apparatus is further configured to determine at least one metric of electrical heterogeneity based on the plurality of activation times.

Embodiment 11. The system as in any one of embodiments 1-10, wherein the electrical activity is representative of depolarization of cardiac tissue that propagates through the torso of the patient.

Embodiment 12. The system as in any one of embodiments 1-11, wherein the plurality of external electrodes comprises a plurality of surface electrodes to be located proximate skin of the patient's torso.

Embodiment 13. A method for use in cardiac evaluation comprising:
monitoring electrical activity from tissue of a patient using a plurality of external electrodes to generate a plurality of electrical signals over time;
filtering the plurality of electrical signals using a first filter having a first frequency range to generate a plurality of first filtered signals;
filtering the plurality of electrical signals using a second filter having a second frequency range different than the first frequency range to generate a plurality of second filtered signals;
detecting at least one QRS complex based on the plurality of first filtered signals; and
detecting a QRS peak based on the plurality of second filtered signals and the detected at least one QRS complex.

Embodiment 14. The method of embodiment 13, further comprising:
determining a threshold function based on the QRS peak; and
detecting at least one subsequent QRS complex based on the threshold function.

Embodiment 15. The method as in any one of embodiments 13-14, further comprising determining a QRS onset time value and a QRS offset time value corresponding to the at least one QRS complex.

Embodiment 16. The method as in any one of embodiments 13-15, wherein determining a QRS onset time value and a QRS offset time value comprises:
generating a dispersion signal from the plurality second filtered signals, wherein the dispersion signal is representative of the dispersion of the plurality second filtered signals over time,
determining a first derivative signal based on the dispersion signal;
determining a first window of the first derivative signal within a first time period before a peak positive value of the first derivative signal;
determining a second window of the first derivative signal within a second time period after a peak negative value of the first derivative signal;
determining the onset time value by determining a last point within the first window that is less than a first threshold; and
determining the offset time value by determining a first point within the second window that is greater than a second threshold.

Embodiment 17. A system for use in cardiac evaluation comprising:
an electrode apparatus comprising a plurality of external electrodes to be disposed proximate a patient's skin; and
a computing apparatus comprising processing circuitry, the computing apparatus operably coupled to the electrode apparatus and configured to:
monitor electrical activity from tissue of a patient using a plurality of external electrodes to generate a plurality of electrical signals over time;
filter the plurality of electrical signals using at least one filter to generate plurality of filtered signals;
detect a QRS peak based on the plurality of filtered signals;
determine a threshold function based on the QRS peak, the threshold function configured to provide sensitivity for detecting at least one subsequent QRS complex; and
detect the at least one subsequent QRS complex based on the threshold function.

Embodiment 18. The method of embodiment 17, wherein the threshold function is configured to decrease from the at least one QRS complex until the at least one subsequent QRS complex is detected or a base threshold is reached.

Embodiment 19. The system of embodiment 18, wherein the base threshold is a base percentage of the QRS peak.

Embodiment 20. The system as in any one of embodiments 17-19, wherein at least a portion of the threshold function has a linear decrease.

Embodiment 21. The system as in any one of embodiments 17-20, wherein the threshold function is configured to linearly decrease from a first threshold to a second threshold, wherein the first threshold is a first percentage of the QRS peak and the second threshold is a second percentage of the QRS peak.

Embodiment 22. The system of embodiment 21, wherein the first percentage is about 60% and the second percentage is about 30%.

Embodiment 23. The system of embodiment 22, wherein the threshold function is configured to stay constant between a time that the second threshold is reached and until a predetermined amount of time after the detected QRS complex.

Embodiment 24. The system of embodiment 23, wherein the threshold function is configured to drop at the predetermined amount of time to a third threshold that is a third percentage of the QRS peak.

Embodiment 25. The system of embodiment 24, wherein the third percentage is about 15%.

Embodiment 26. The system of embodiment 24, wherein the threshold function is configured to drop linearly from the third threshold to a base threshold.

Embodiment 27. The system of embodiment 26, wherein the base percentage is about 3%.

Embodiment 28. A method for use in cardiac evaluation comprising:
monitoring electrical activity from tissue of a patient using a plurality of external electrodes to generate a plurality of electrical signals over time;
filtering the plurality of electrical signals using at least one filter to generate a plurality of filtered signals;
detecting a QRS peak based on the plurality of filtered signals.
determining a threshold function based on the QRS peak, the threshold function configured to provide sensitivity for detecting at least one subsequent QRS complex; and
detecting the at least one subsequent QRS complex based on the threshold function.

Embodiment 29. The method of embodiment 28, wherein the threshold function is configured to decrease from the at least one QRS complex until the at least one subsequent QRS complex is detected or a base threshold is reached.

Embodiment 30. The system of embodiment 29, wherein the base threshold is a base percentage of the QRS peak.

Embodiment 31. A system for use in cardiac evaluation comprising:
an electrode apparatus comprising a plurality of external electrodes to be disposed proximate a patient's skin; and
a computing apparatus comprising processing circuitry, the computing apparatus operably coupled to the electrode apparatus and configured to:
monitor electrical activity from tissue of a patient using a plurality of external electrodes to generate a plurality of electrical signals over time;
filter the plurality of electrical signals using a first filter and a second filter to generate a plurality of first filtered signals and plurality of second filtered signals, the first filter and the second filter having different frequency ranges;
detect a QRS peak based on the plurality of first and second filtered signals.
generate a dispersion signal from the plurality second filtered signals, wherein the dispersion signal is representative of the dispersion of the plurality second filtered signals over time,
determine a first derivative signal based on the dispersion signal;
determine a QRS onset time value and a QRS offset time value corresponding to the at least one QRS complex based on the first derivative signal.

Embodiment 32. The system of embodiment 31, wherein determining the QRS onset time value and the QRS offset time value comprises:
determining a first window of the first derivative signal within a first time period before a peak positive value of the first derivative signal;
determining a second window of the first derivative signal within a second time period after a peak negative value of the first derivative signal;
determining the onset time value by determining a last point within the first window that is less than a first threshold; and
determining the offset time value by determining a first point within the second window that is greater than a second threshold.

Embodiment 33. The system as in any one of embodiments 31-32, wherein the computing apparatus is further configured to determine an activation time for the at least one QRS complex within each of the plurality of cardiac signals within a QRS duration between the QRS onset time value and the QRS offset time value.

Embodiment 34. The system of embodiment 33, wherein the computing apparatus is further configured to determine at least one metric of electrical heterogeneity based on the activation time for each of the at least one QRS complex within each of the plurality of cardiac signals.

This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

What is claimed:

1. A system for use in cardiac evaluation comprising:
an electrode apparatus comprising a plurality of external electrodes to be disposed proximate a patient's skin; and
a computing apparatus comprising processing circuitry, the computing apparatus operably coupled to the electrode apparatus and configured to:
monitor electrical activity from tissue of a patient using a plurality of external electrodes to generate a plurality of electrical signals over time;
filter the plurality of electrical signals using at least one filter to generate plurality of filtered signals;
detect a QRS peak based on the plurality of filtered signals;
determine a threshold function based on the QRS peak, the threshold function configured to provide sensitivity for detecting at least one subsequent QRS complex; and
detect the at least one subsequent QRS complex based on the threshold function.

2. The system of claim 1, wherein the threshold function is configured to decrease from the at least one QRS complex until the at least one subsequent QRS complex is detected or a base threshold is reached.

3. The system of claim 2, wherein the base threshold is a base percentage of the QRS peak.

4. The system of claim 1, wherein at least a portion of the threshold function has a linear decrease.

5. The system of claim 1, wherein the threshold function is configured to linearly decrease from a first threshold to a second threshold, wherein the first threshold is a first percentage of the QRS peak and the second threshold is a second percentage of the QRS peak.

6. The system of claim 5, wherein the first percentage is about 60% and the second percentage is about 30%.

7. The system of claim 6, wherein the threshold function is configured to stay constant between a time that the second threshold is reached and until a predetermined amount of time after the detected QRS complex.

8. The system of claim 7, wherein the threshold function is configured to drop at the predetermined amount of time to a third threshold that is a third percentage of the QRS peak.

9. The system of claim 8, wherein the third percentage is about 15%.

10. The system of claim 8, wherein the threshold function is configured to drop linearly from the third threshold to a base threshold.

11. The system of claim 10, wherein the base percentage is about 3%.

12. A method for use in cardiac evaluation comprising:
monitoring electrical activity from tissue of a patient using a plurality of external electrodes to generate a plurality of electrical signals over time;
filtering the plurality of electrical signals using at least one filter to generate a plurality of filtered signals;
detecting a QRS peak based on the plurality of filtered signals.
determining a threshold function based on the QRS peak, the threshold function configured to provide sensitivity for detecting at least one subsequent QRS complex; and
detecting the at least one subsequent QRS complex based on the threshold function.

13. The method of claim 12, wherein the threshold function is configured to decrease from the at least one QRS complex until the at least one subsequent QRS complex is detected or a base threshold is reached.

14. The system of claim 13, wherein the base threshold is a base percentage of the QRS peak.
